SEQUENCE LISTING (4 sequences)

SEQ ID NO: 1 - DNA, 52 bp, Artificial Sequence
  Primer
tcgtcggcagcgtcagatgtgtataagagacaggtgccagcmgccgcggtaa

SEQ ID NO: 2 - DNA, 54 bp, Artificial Sequence
  Primer
gtctcgtgggctcggagatgtgtataagagacagggactachvgggtwtctaat

SEQ ID NO: 3 - DNA, 17 bp, Artificial Sequence
  Primer
ggcaagtgttcttcgga

SEQ ID NO: 4 - DNA, 17 bp, Artificial Sequence
  Primer
ggctcaaccctggacag

(19) United States
(12) Patent Application Publication
(10) Pub. No.: US 2022/0264890 A1
(43) Pub. Date: Aug. 25, 2022

(54) Novel Bacterial Strains

(71) Applicant: Agriculture Victoria Services PTY LTD, Bundoora, Victoria (AU)

(72) Inventors: Jatinder Kaur, Taylors Hill (AU); Ian Ross Tannenbaum, Bundoora (AU); Tongda Li, Southbank (AU); Timothy Ivor Sawbridge, Coburg (AU); Ross Mann, Coburg (AU); German Carlos Spangenberg, Bundoora (AU); Ankush Chanel, Bundoora (AU); Guodong Yang, Luoyang City (CN); Holly Hone, Parkville (AU)

(73) Assignee: Agriculture Victoria Services PTY LTD, Bundoora, Victoria (AU)

(21) Appl. No.: 17/627,763

(22) PCT Filed: Jul. 17, 2020
(86) PCT No.: PCT/AU2020/050734

§ 371 Date: Jan. 17, 2022

(30) Foreign Application Priority Data
Jul. 19, 2019 (AU) 2019902564

(51) Int. Cl. A01N 63/20; C12N 1/20

(52) U.S. Cl. CPC A01N 63/20; C12N 1/205; C12R 2001/06

(57) ABSTRACT

The present invention relates to a substantially purified or isolated endophyte strain from a plant, wherein said endophyte is a strain of Curtobacterium laccumfaciens and/or Arthrobacter sp., and wherein said endophyte provides improved environmental stress tolerance to plants into which it is inoculated. The present invention also relates to plants infected with the endophytes and related methods.

### FIELD OF THE INVENTION

The present invention relates to novel bacterial microbiome strains, plants infected with such and related methods.

### BACKGROUND OF THE INVENTION

Microbes represent an invaluable source of novel genes and compounds that have the potential to be utilised in a range of industrial sectors. Scientific literature gives numerous accounts of microbes being the primary source of antibiotics, immune-suppressants, anticancer agents and cholesterol-lowering drugs, in addition to their use in environmental decontamination and in the production of food and cosmetics.

A relatively unexplored group of microbes known as endophytes, which reside e.g. in the tissues of living plants, offer a particularly diverse source of novel compounds and genes that may provide important benefits to society, and in particular, agriculture.

Endophytes may be fungal or bacterial. Endophytes often form mutualistic relationships with their hosts, with the endophyte conferring increased fitness to the host, often through the production of defence or other beneficial compounds. At the same time, the host plant offers the benefits of a protected environment and nutriment to the endophyte.

Important grain crops, such as wheat (Triticum aestivum), are commonly found in association with fungal and bacterial endophytes. However, there remains a general lack of information and knowledge of these endophytes as well as of methods for the identification and characterisation of novel endophytes and their deployment in plant improvement programs.

Knowledge of the endophytes of these crops may allow certain beneficial traits to be exploited, or lead to other agricultural advances, e.g. to the benefit of sustainable agriculture and the environment.

There exists a need to overcome, or at least alleviate, one or more of the difficulties or deficiencies associated with the prior art.

### SUMMARY OF THE INVENTION

In one aspect, the present invention provides a substantially purified or isolated endophyte strain from a plant, wherein said endophyte is a strain of Curtobacterium flaccumfaciens and/or Arthrobacter sp., and wherein said endophyte strain provides improved environmental stress tolerance to plants into which it is inoculated.

In a preferred embodiment, the Curtobacterium flaccumfaciens strain may be a strain selected from the group consisting of D3-27 and D3-25 as described herein and as deposited with the National Measurement Institute of 1/153 Bertie Street, Port Melbourne, VIC 3207, Australia on 9 Jul. 2019 with accession numbers V19/013682 and V19/013683, respectively.

In another preferred embodiment, the Arthrobacter sp. strain may be a strain selected from the group consisting of D4-11, D4-14, and D4-55 as described herein and as deposited with the National Measurement Institute of 1/153 Bertie Street, Port Melbourne, VIC 3207, Australia on 9 Jul. 2019 with accession numbers V19/013680, V19/013681 and V19/013684, respectively.

As used herein the term “endophyte” is meant a bacterial or fungal strain that is closely associated with a plant. By “associated with” in this context is meant that the bacterium or fungus lives on, in or in close proximity to a plant. For example, it may be endophytic, for example living within the internal tissues of a plant, or epiphytic, for example growing externally on a plant.

The endophyte may provide a beneficial phenotype in the plant harbouring, or otherwise associated with the endophyte.

By “environmental stress tolerance” as used herein means that the endophyte possesses genetic and/or metabolic characteristics that result in improved tolerance to stress conditions in a plant harbouring, or otherwise associated with, the endophyte. Such improved tolerance to environmental stress conditions include improved resistance to pests and/or diseases (including fungal and bacterial pathogens), improved tolerance to water and/or nutrient stress, enhanced biotic stress tolerance, enhanced drought tolerance, enhanced water use efficiency, reduced toxicity and enhanced vigour in the plant with which the endophyte is associated, relative to an organism not harbouring the endophyte or harbouring a control endophyte such as standard toxic (ST) endophyte.

Environmental stress to the plant may be caused by exposure to abiotic and/or biotic conditions. Abiotic stress conditions are non-biological factors including drought, salinity, heat, cold and pollution (e.g. heavy metals in soil).

Preferably, environmental stress tolerance is drought stress tolerance. Thus, the endophyte provides the plant with which it is associated with improved drought tolerance and/or resistance to drought conditions. Drought conditions result from below-average rainfall or precipitation in a region that leads to water reduced water supply. Drought conditions can also be enhanced or prolonged by other weather extremes such as temperature.

Alternatively, or in addition, the endophyte may be suitable as a biofertilizer to improve tolerance to abiotic stresses, in particular drought tolerance and/or resistance to a plant. That is, the endophyte may provide protection to the plant against the effects of abiotic stress conditions, such as drought.

Alternatively, or in addition, the endophyte may be suitable as a biostimulant. That is, the endophyte may improve or stimulate growth of the plant (i.e. growth promotion). The biostimulant activity may also improve or stimulate growth of a plant under abiotic stress conditions.

As used herein the term “substantially purified” is meant that an endophyte is free of other organisms. The term includes, for example, an endophyte in axenic culture. Preferably, the endophyte is at least approximately 90% pure, more preferably at least approximately 95% pure, even more preferably at least approximately 98% pure, even more preferably at least approximately 99% pure.

As used herein the term ‘isolated’ means that an endophyte is removed from its original environment (e.g. the natural environment if it is naturally occurring). For example, a naturally occurring endophyte present in a living plant is not isolated, but the same endophyte separated from some or all of the coexisting materials in the natural system, is isolated.

Preferably, the endophyte is purified or isolated from a plant of the Poaceae family, particularly from the genus Triticum, including T. aestivium.

Preferably, the endophyte is purified or isolated from a plant that is tolerant to abiotic conditions, more particularly, drought tolerant.

Phytohormones are important growth regulators synthesized in defined organs of a plant that have a prominent impact on plant metabolism and are considered to play an important role in the mitigation of abiotic stresses, such as auxins, cytokinins, gibberellic acid (GA), abscisic acid (ABA), jasmonic acid and salicylic acid (SA). However, abiotic stresses alter the endogenous levels of phytohormones. Abiotic stress, such as drought may inhibit phytohormone concentrations in plant tissue.

Without wishing to be bound by theory, the endophyte may provide a source of phytohormone to the plant, particularly when under abiotic stress. The phytohormone synthesising ability of the endophyte plays a role in providing protection or improved responses to abiotic stresses, such as drought and in addition to enhanced growth, in host plants.

Thus, in one aspect of the invention, the endophyte produces phytohormones, such as auxins, cytokinins, gibberellins, abscisic acid (ABA), jasmonic acid and salicylic acid (SA). Preferably, the phytohormone is an auxin. More preferably, the auxin is indole acetic acid (IAA).

Preferably, the endophyte may also be non-pathogenic. That is, when associated with a non-pathogenic endophyte the plant does not exhibit disease symptoms. Without wishing to be bound by theory, this may be due to the endophyte having a reduced complement or otherwise absence of genes associated with pathogenicity, such as virulence regulators (e.g. slyA), secretions systems (e.g. (Type II, Type IV), ABC transporters (Lipopolysaccharides, phosphonates), carbohydrate metabolism (Fructose/Mannose; Starch/Sucrose). Preferably, the virulence regulation genes and/or ABC transporter genes are absent.

Also in preferred embodiments, the plant or part thereof includes an endophyte-free host plant or part thereof stably infected with said endophyte.

The plant inoculated with the endophyte may be a grass or non-grass plant suitable for agriculture, specifically a forage, turf, or bioenergy grass, or a grain crop or industrial crop.

The forage, turf or bioenergy grass may include for example, those belonging to the genera Lolium and Festuca, including L. perenne (perennial ryegrass) and L. arundinaceum (tall fescue) and L. multiflorum (Italian ryegrass).

The grain crop may be a non-grass species, for example, any of soybeans, cotton and grain legumes, such as lentils, field peas, fava beans, lupins and chickpeas, as well as oilseed crops, such as canola.

Thus, the grain crop or industrial crop species may selected from the group consisting of wheat, barley, oats, chickpeas, triticale, fava beans, lupins, field peas, canola, cereal rye, vetch, lentils, millet/panicum, safflower, linseed, sorghum, sunflower, maize, canola, mungbeans, soybeans, bean (e.g. snapbean) and cotton.

The grain crop or industrial crop grass may be those belonging to the genus Triticum, including T. aestivum (wheat), those belonging to the genus Avena, including A. sativa (oats), those belonging to the genus Hordeum, including H. vulgare (barley), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), and those belonging to the genera Miscanthus, Paspalum, Pennisetum, Poa, Eragrostis and Agrostis. Preferably, the plant belongs to the genus Triticum, more preferably, the plant is T. aestivium (wheat).

A plant or part thereof may be infected by a method selected from the group consisting of inoculation, breeding, crossing, hybridisation, transduction, transfection, transformation and/or gene targeting and combinations thereof.

The part thereof of the plant may be, for example, a seed.

Without wishing to be bound by theory, it is believed that the endophyte of the present invention may be transferred through seed from one plant generation to the next. The endophyte may then spread or locate to other tissues as the plant grows, i.e. to roots. Alternatively, or in addition, the endophyte may be recruited to the plant root, e.g. from soil, and spread or locate to other tissues.

Thus, in a further aspect, the present invention provides a plant, plant seed or other plant part derived from a plant or part thereof as hereinbefore described. In preferred embodiments, the plant, plant seed or other plant part may produce phytohormone. Preferably, the phytohormone is an auxin, more preferably IAA or derivative, isomer and/or salt thereof.

In another aspect, the present invention provides the use of an endophyte as hereinbefore described to produce a plant or part thereof stably infected with said endophyte. The present invention also provides the use of an endophyte as hereinbefore described to produce a plant or part thereof as hereinbefore described.

In another aspect, the present invention provides a phytohormone produced by an endophyte as hereinbefore described, or a derivative, isomer and/or a salt thereof. Preferably, the phytohormone is an auxin, more preferably IAA or derivative, isomer and/or salt thereof.

The phytohormone may be produced by the endophyte when associated with the plant. Thus, in another aspect, the present invention provides a method for producing a phytohormone, said method including infecting a plant with an endophyte as hereinbefore described and cultivating the plant under conditions suitable to produce the phytohormone. The endophyte-infected plant or part thereof may be cultivated by known techniques. The person skilled in the art may readily determine appropriate conditions depending on the plant or part thereof to be cultivated. Preferably, the phytohormone is an auxin, more preferably IAA or derivative, isomer and/or salt thereof.

The phytohormone may also be produced by the endophyte when it is not associated with a plant. Thus, in yet another aspect, the present invention provides a method for producing a phytohormone, said method including culturing an endophyte as hereinbefore described, under conditions suitable to produce the phytohormone. Preferably, the phytohormone is an auxin, more preferably IAA or derivative, isomer and/or salt thereof.

In a preferred embodiment of this aspect of the invention, the method may include the further step of isolating the phytohormone compound from the plant or culture medium.

The endophyte-infected plant or part thereof may be cultivated by known techniques. The person skilled in the art may readily determine appropriate conditions depending on the plant or part thereof to be cultivated. In some embodiments, the plant or plant part is cultivated under environmental stress conditions, preferably drought and/or rainfed conditions.

The production of a phytohormone has particular utility in agricultural plant species, in particular, forage, turf, or bioenergy grass species, or grain crop species or industrial crop species. These plants may be cultivated across large areas of e.g. soil where the properties and biological processes of the endophyte as hereinbefore described and/or phytohormone compound produced by the endophyte may be exploited at scale.

In preferred embodiments, the endophyte may be a Curtobacterium flaccumfaciens strain selected from the group consisting of D3-27 and D3-25 as described herein and as deposited with the National Measurement Institute of 9 Jul. 2019 with accession numbers V19/013682 and V19/013683, respectively.

In another preferred embodiment, the endophyte may be a Arthrobacter sp. strain selected from the group consisting of D4-11, D4-14, and D4-55 as described herein and as deposited with the National Measurement Institute of 9 Jul. 2019 with accession numbers V19/013680, V19/013681 and V19/013684, respectively.

Preferably, the plant is a forage, turf, bioenergy grass species or grain crop or industrial crop species, as hereinbefore described.

In another aspect, the present invention provides a method of stimulating growth of a plant or plant part, said method including:
infecting said plant or plant part with an endophyte as hereinbefore described; and
cultivating infected plant or plant part.

Preferably, the plant or plant part is a seed.

Preferably, the plant or plant part is an agricultural plant species selected from one or more of forage grass, turf grass, bioenergy grass, grain crop and industrial crop.

Preferably, the plant into which the endophyte is inoculated is a grain crop or industrial crop grass selected from the group consisting of those belonging to the genus Triticum, including T. aestivum (wheat), those belonging to the genus Avena including A. sativa (oats) those belonging to the genus Hordeum, including H. vulgare (barley), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), those belonging to the genera Miscanthus, Paspalum, Pennisetum, Poa, Eragrostis and Agrostis,

Preferably, the inoculated plant or plant part is cultivated under environmental stress conditions, preferably drought conditions.

In another aspect, a method for enriching a plant or plant part for endophytes conferring improved environmental stress tolerance, said method including:
cultivating plant or plant parts under environmental stress conditions;
measuring plant or plant parts to identify germplasm that is tolerant or susceptible to environmental stress conditions;
profiling and isolating endophytes from a plant or plant part that is tolerant or susceptible to environmental stress conditions
identifying endophytes enriched in a plant or plant part that is tolerant to environmental stress conditions;
infecting said plant or plant part with germplasms of identified endophytes to confer improved environmental stress tolerance.

Preferably, the environmental stress tolerance is drought tolerance.

The plant or part thereof may be cultivated under environmental stress conditions, preferably drought conditions. For example, the plant or plant parts may be cultivated under different watering conditions: well-watered (300 mL water every two days), mild drought (150 mL of water every two days), or severe drought (50 mL every two days).

Measuring drought tolerance or susceptibility may include, for example, calculation of a drought susceptibility index (DSI) for plant or plant part cultivated under drought and rainfed conditions, based on the difference in performance under drought and rainfed conditions.

Preferably, the plant or plant part is a seed.

Preferably, the plant or plant part is an agricultural plant species selected from one or more of forage grass, turf grass, bioenergy grass, grain crop and industrial crop.

Preferably, the plant into which the endophyte is inoculated is a grain crop or industrial crop grass selected from the group consisting of those belonging to the genus Triticum, including T. aestivum (wheat), those belonging to the genus Avena including A. sativa (oats) those belonging to the genus Hordeum, including H. vulgare (barley), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), those belonging to the genera Miscanthus, Paspalum, Pennisetum, Poa, Eragrostis and Agrostis.

Preferably, the endophyte is an endophyte as hereinbefore described.

In this specification, the term ‘comprises’ and its variants are not intended to exclude the presence of other integers, components or steps.

In this specification, reference to any prior art in the specification is not and should not be taken as an acknowledgement or any form of suggestion that this prior art forms part of the common general knowledge in Australia or any other jurisdiction or that this prior art could reasonably expected to be combined by a person skilled in the art.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE DRAWINGS/FIGURES

In the Figures:

FIG. 1—Yield and drought susceptibility index (DSI) of wheat lines under drought (rainout shelter) and rainfed conditions to determine if they are drought tolerant or susceptible. The stars represent wheat lines selected for microbiome profiling (Drought tolerant: lines 1 to 4; Drought susceptible: lines 9, 10, and 11).

FIG. 2—PCA (Robust Aitchison) of the microbiomes of drought tolerant lines (white circles—Drought tolerant lines 1, 2, 3, and 4) and drought susceptible lines (grey circles—Drought susceptible lines 9, 10 and 11 ).

FIG. 3—Venn diagram of the number of OTUs associated with drought tolerant lines (mid grey circle—lines 1, 2, 3, and 4) and drought susceptible lines (light grey circle—lines 9, 10 and 11).

FIG. 4—The OTU Curtobacterium flaccumfaciens in drought tolerant and drought susceptible lines, grown under drought and rainfed conditions. The Y-axis represents the percentage of the total number of reads.

FIG. 5—The OTU Arthrobacter sp. in drought tolerant and drought susceptible lines, grown under drought and rainfed conditions. The Y-axis represents the percentage of the total number of reads.

FIG. 6—Protein spectra of six representative Curtobacterium flaccumfaciens strains (FIG. 6A: D3-47, FIG. 6B D3-32, FIG. 6C D3-25, FIG. 6D: D3-34, FIG. 6E: D3-27, FIG. 6F: D3-19) from the unique Curtobacterium clade.

FIG. 7—Protein spectra of six representative Arthrobacter sp. strains (FIG. 7A: D4-8, FIG. 7B: D4-11, FIG. 7C: D4-14, FIG. 7D: D4-21, FIG. 7E: D4-25 and FIG. 7F: D4-55) from the unique Arthrobacter clade.

FIG. 8-FIG. 8A. ANI phylogram and associated heatmap of 124 Curtobacterium spp. strains including novel Curtobacterium flaccumfaciens strain D3-25 (indicated with a star). FIG. 8B. Inset of a clade within the ANI phylogram containing the novel Curtobacterium flaccumfaciens strain D3-25 (indicated with a star) and closely related species. FIG. 8C A Table from left to right: detail of Key to heatmap with percentage intensity shown in top left of FIG. 8A; list of strains in Section 1 located at the top of the Y-axis shown in FIG. 8A; list of strains in Section 2 located in the middle of the Y-axis shown in FIG. 8A; list of strains in Section 3 located at the bottom of the Y-axis shown in FIG. 8A (these strains also correlate to those shown in the details of FIG. 8B.

FIG. 9—Phylogeny of Arthrobacter sp. and novel Arthrobacter sp. strains D4-11, D4-14 and D4-55. The maximum-likelihood tree was inferred based on 118 genes conserved among 8 genomes. Values shown next to branches were the local support values calculated using 1000 resamples with the Shimodaira-Hasegawa test.

FIG. 10—In vitro bioassay assessing the ability of Curtobacterium flaccumfaciens novel strains D3-25 and D3-27 and Arthrobacter sp. novel strains D4-11, D4-14 and D4-55 to produce IAA using the Salkowski method, compared to IAA standards (0, 5, 10, 20, 50 and 100 μg/mL). Corresponding ppb is provided in Table 4.

FIG. 11—Average shoot length of 4-week old wheat seedlings inoculated with Curtobacterium flaccumfaciens novel strains D3-25 and D3-27, Arthrobacter sp. novel strains D4-11, D4-14 and D4-55, and a related bacterial strain Bac1. The * indicates significant difference in the mean at p≤0.05 between the control and the bacterial strains.

FIG. 12—Image of 4 week old bean seedlings inoculated with Curtobacterium flaccumfaciens novel strains D3-25 and D3-27, Arthrobacter sp. novel strains D4-11, D4-14 and D4-55 and an untreated control.

FIG. 13—Average root length of 7 day old wheat, oat, ryecorn and barley seedlings inoculated with Curtobacterium flaccumfaciens novel strains D3-25, Arthrobacter sp. novel strains D4-14. The * indicates significant difference in the mean at p≤0.05 between the control and the bacterial strains.

FIG. 14—Average shoot length of 6 week old wheat seedlings inoculated with Curtobacterium flaccumfaciens novel strains D3-25, Arthrobacter sp. novel strains D4-14, under well-watered, mild drought and severe drought conditions. The * indicates significant difference in the mean at p≤0.05 between the control and the bacterial strains.

FIG. 15—Average root length of 6 week old wheat seedlings inoculated with Curtobacterium flaccumfaciens novel strains D3-25, Arthrobacter sp. novel strains D4-14, under well-watered, mild drought and severe drought conditions. The * indicates significant difference in the mean at p 0.05 between the control and the bacterial strains.

### DETAILED DESCRIPTION OF THE EMBODIMENTS

Novel plant associated Curtobacterium flaccumfaciens and Arthrobacter sp. bacterial strains have been isolated from wheat (Triticum aestivum) plants. The novel bacterial strains were identified in the seed microbiome of wheat and were particularly associated with drought tolerant lines under drought stress conditions. The novel bacterial strains were isolated from seeds of drought tolerant wheat lines. The genome of the Curtobacterium flaccumfaciens and Arthrobacter sp. bacterial strains were sequenced and are shown to be novel, related to Curtobacterium flaccumfaciens and Arthrobacter sp. (YN) respectively. Analysis of the genome sequence has shown that the Curtobacterium flaccumfaciens and Arthrobacter sp. novel bacterial strains have genes associated with phytohormone production (indole acetic acid, IAA) and environmental stress tolerance, along with an absence of genes associated with pathogenicity (virulence regulators, secretion systems, ABC transporters, carbohydrate metabolism). In vitro assays indicate that the Arthrobacter sp. strains produce the phytohormones IAA. In planta assays indicate that the novel Curtobacterium flaccumfaciens and Arthrobacter sp. strains increase early vigour in wheat seedlings and are non-pathogenic on wheat and bean.

### EXAMPLE 1

### Assessment of Drought Tolerance and Susceptibility in 11 Wheat Lines

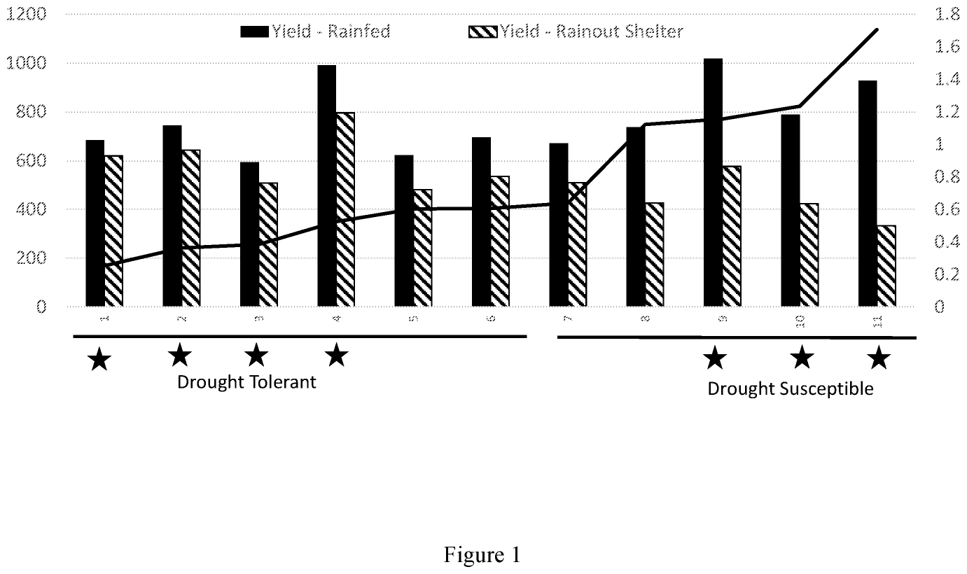

A field trial was established to evaluate the drought tolerance and susceptibility of 11 wheat lines, under drought and rainfed conditions. There were a total of 3 replicates per line for both drought and rainfed conditions. Drought conditions were created by planting wheat lines under a rainout shelter, and exposing the plants to precipitation events consistent with a season designated as under drought. Rainfed conditions were created by planting wheat lines adjacent to the rainout shelter and exposing the plants to natural precipitation events. The wheat lines were cultivated and harvested according to standard practices (i.e. drilling depth, fertiliser regime, row spacing, etc.). Wheat yields were calculated for all lines under drought and rainfed conditions, and a drought susceptibility index (DSI) was calculated for each line, based on the difference in performance under drought and rainfed conditions. The wheat lines were categorised as either drought tolerant or susceptible based on the DSI (FIG. 1).

### EXAMPLE 2

### Microbiome Profiling of Drought Tolerant/Susceptible Wheat Lines to Determine Bacterial OTUs Associated With Drought Tolerance

The microbiome of 7 wheat lines was profiled, including 4 drought tolerant lines (lines 1, 2, 3, and 4) and 3 drought susceptible lines (lines 9, 10 and 11). Seeds from each line were plated onto three stacked pieces of sterile filter paper soaked in sterile distilled water. These seeds were then allowed to germinate at room temperature in the dark for two days. The plates were then moved into light conditions and seedlings allowed to grow for a further four days. The seedlings were harvested and the seed husks discarded. Plant tissues from 5 seedlings were pooled constituting one replicate. Each line had 10 replicates. Each replicate was snap frozen in liquid nitrogen. DNA extraction of replicates was performed in 96-well plates using the QIAGEN MagAttract 96 DNA Plant Core Kit according to manufacturers' instructions, with minor modifications to include the use of a Biomek FX liquid handling station. The bacterial microbiome was profiled targeting the V4 region of the 16S rRNA gene according to the IIlumina 16S Metagenomic Sequencing Library Preparation protocol, with minor modifications to include the use of PNA blockers to reduce host organelle amplification (Wagner et al, 2016). In brief, the V4 region was amplified using the following reagents: 12.5 μL 2X KAPA HiFi HotStart ReadyMix, 5 μL of each of the 515F with adapter (5′-TCGTCGGCAGCGTCAGATGTGTATAAGAGACAG/GTGCCAGCMGCCGCGGTAA-3′) (SEQ ID NO: 1) and 806R (5′-GTCTCGTGGGCTCGGAGATGTGTATAAGAGACAG/GGACTACHVGGGTWTCTAAT-3′) (SEQ ID NO: 2) primers, 5 μL of 50 nM mP01 (GGCAAGTGTTCTTCGGA) (SEQ ID NO: 3) and 5 μL of 50 nM pP01 (GGCTCAACCCTGGACAG) (SEQ ID NO: 4) PNA blockers, and 2.5 μL 5 ng/μL of Template DNA to a final volume of 25 μL. The PCR reaction was run in an Agilent Surecylcer 8800 with the following conditions: denaturation at 95° C. for 3 min; 25 cycles of 94° C. for 30 sec, 75° C. for 10 sec, 55° C. for 10 sec, 72° C. for 30 sec; and one final extension at 72° C. for 5 min. The V4 region PCR amplicons were purified using AMPure XP beads and indexed using the following reagents: 5 μL of the purified PCR amplicons, 5 μL Nextera XT Index Primer 1 (N7xx), 5 μL Nextera XT Index Primer 2 (SSxx), 25 μL 2X KAPA HiFi HotStart ReadyMix, and 10 μL sterile water. The PCR was run in an Agilent Surecylcer 8800 with the following conditions: denaturation at 95° C. for 3 min; 8 cycles of 95° C. for 30 sec, 55° C. for 30 sec, and 72° C. for 30 sec; and one final extension at 72° for 5 min. The PCR amplicon was again purified using AMPure XP beads. Libraries were quantified, pooled, requantified (NanoDrop and Qubit) and loaded at a final concentration of between 0.5-20 nM. Paired-end sequencing was performed on HiSeq3000 using a 2×150 bp v3 chemistry cartridge. Sequence data was trimmed and paired using PandaSEQ (Massela et al., 2012). Operational Taxonomic Unit (OTU) picking and counting, dereplication and denoising, and taxonomical assignment was performed using custom scripts or QIIME2 (release 2019.1). Comparative analysis of treatments was conducted using QIIME2 or Genedata Expressionist, Analyst Module (Genedata AG, Basel, Switzerland).

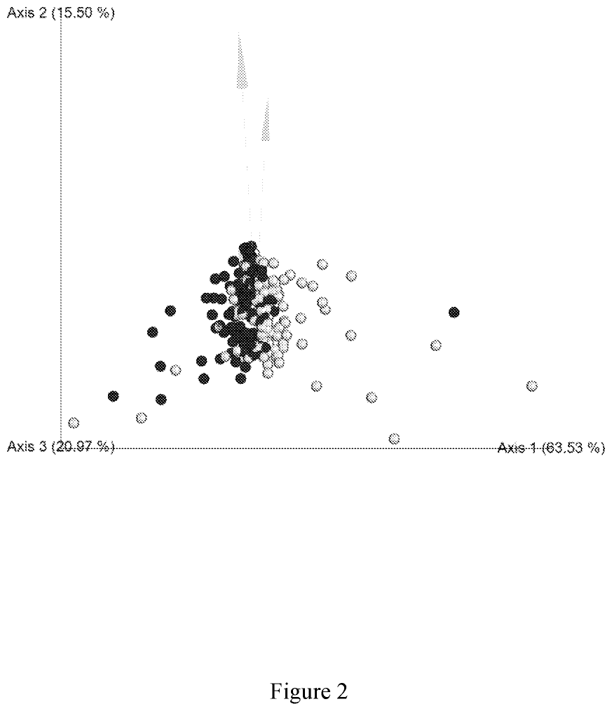

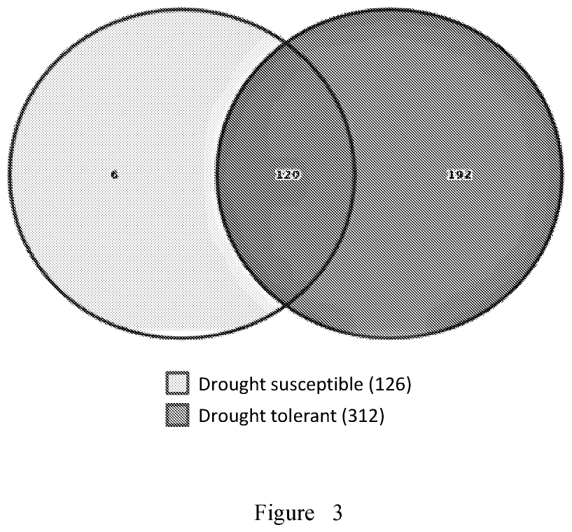

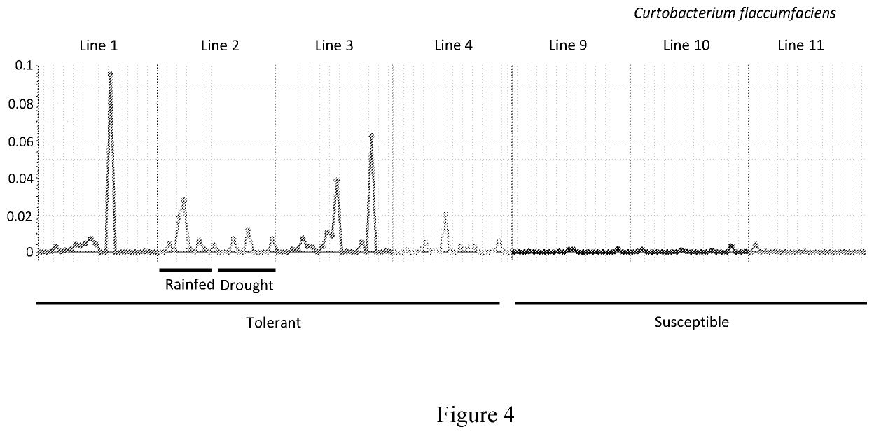

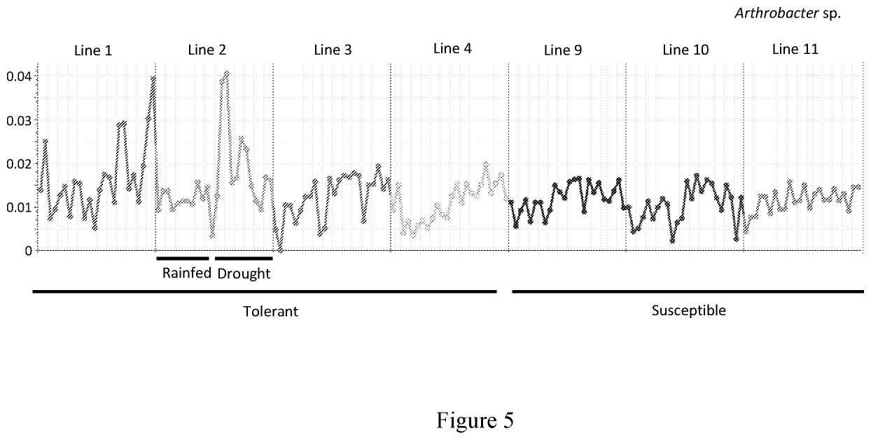

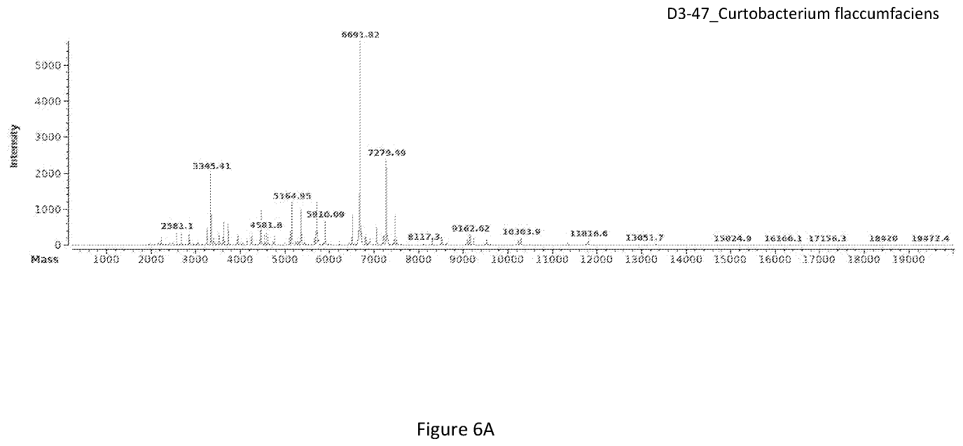

The microbiome profiles of drought tolerant lines (line 1, 2, 3, and 4) were different from drought susceptible lines (FIG. 2). Drought tolerant lines had higher microbial diversity than drought susceptible lines (FIG. 3). OTUs were identified that showed differential concentrations between drought tolerant and drought susceptible lines, and drought and rainfed conditions. The OTU Curtobacterium flaccumfaciens was identified almost exclusively in drought tolerant lines (FIG. 4). A number of OTUs were identified that were higher under drought conditions and lower under rainfed conditions, including the OTUs Arthrobacter sp., Amycolatopsis sp., Acidobacterium sp., Bradyrhizobium sp., Caulobacter sp, Flavobacterium sp., Skermanella sp., Sphingomonas sp., Limnohabitans sp., Commomonas sp., Xylophilus sp., Janthinobacterium sp., Massilia sp., Pseudomonas fluorescens and Pseudomonas sp. (FIG. 5).

### EXAMPLE

### 3

### Isolation of Bacterial Strains From Drought Tolerant and Drought Susceptible Wheat Lines

### Seed Associated Bacterial Strains

Bacteria were isolated from the seeds of two drought tolerant wheat (Triticum aestivum) lines (lines 3 and 4) and two drought susceptible wheat lines (lines 10 and 11) that had been subjected to drought and rainfed conditions. Ten seeds from each treatment were then plated onto three stacked pieces of sterile filter paper soaked in Nystatin 50 ppm. These seeds were then allowed to germinate at room temperature in the dark for two days. The plates were then moved into light conditions and seedlings allowed to grow for a further four days. The seedlings were harvested and the seed husks discarded. Aerial and root tissue from two seedlings from each condition were collected. Plant tissues from the pooled seedlings were then immersed in phosphate buffer solution (PBS) and ground using a Mixermill for up to 1 minute at 30 Hertz. A 10 μl aliquot of the resulting macerate was added to 90 μl of PBS. Further 1:10 dilutions were performed to generate 10⁻³ and 10⁻⁴ solutions. Reasoners 2 Agar (R2A) agar plates were then inoculated with 10⁻³ and 10⁻⁴ solutions from each treatment and allowed to grow for 24-48 hours. Individual bacterial colonies were then streaked onto single R2A plates to isolate single bacterial strains. A total of around 480 bacterial strains were isolated from the four wheat lines and stored in 20% glycerol at -80° C.

### EXAMPLE 4

### Identification of Curtobacterium flaccumfaciens and Arthrobacter sp. Novel Bacterial Strains

### Proteomics

Bacteria were identified using the Bruker MALDI Biotyper system. The bacterial strains were removed from −80° C. glycerol stock and streaked on R2A plates and grown for 48 hours. Single colonies were then prepared for analysis using the Extended Direct Transfer (EDT) method. Escherichia coli strain ATCC 25922 was included as a quality control strain and an internal standard. Each colony was inoculated onto a primary and secondary spot on the Bruker MALDI Biotyper target plate, treated with 70% formic acid for 30 mins and then consequently treated with HCCA matrix solution [10 mg HCAA in 1 mL of solvent solution: 50% volume μL ACN (acetonitrile), 47.5% volume μL water, and 2.5% volume μL TFA (trifluoroacetic acid)], before being allowed to dry at room temperature. The target plate was then analysed by Bruker MALDI-TOF ultrafleXtreme in accordance with the manufacturer's instructions. Protein spectra were calibrated using Escherichia coli ATCC 25922 and the raw spectral data was generated using MALDI BioTyper automation 2.0 software using default settings. Preliminary identification was then determined by comparing protein spectra from unknown strains against known spectra in the MALDI BioTyper library (spectra for 2,750 species from 471 genera—January, 2019). Protein spectra from each novel bacterial strain were passed through a data refinement pipeline in the software Refiner (GeneData). The refined protein spectra were then compared to all strains using the hierarchical clustering algorithm in the software Analyst (GeneData). A phenogram was generated whereby novel bacterial strains clustered based on similar protein profiles.

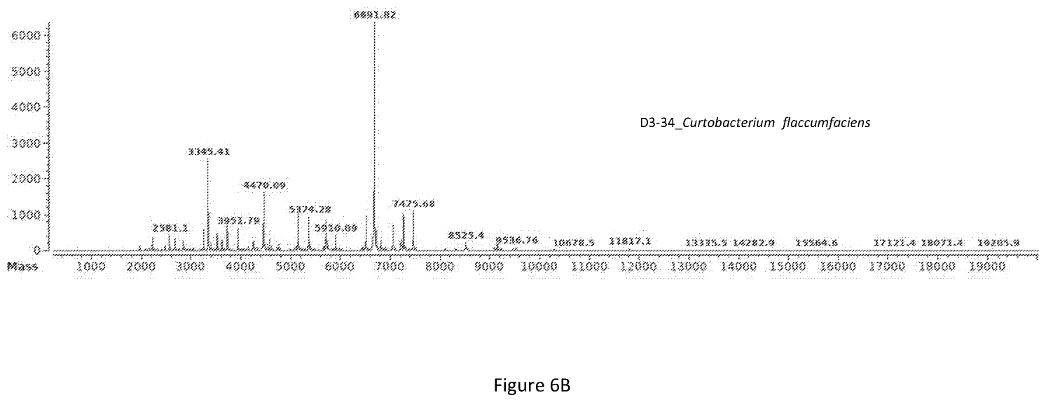

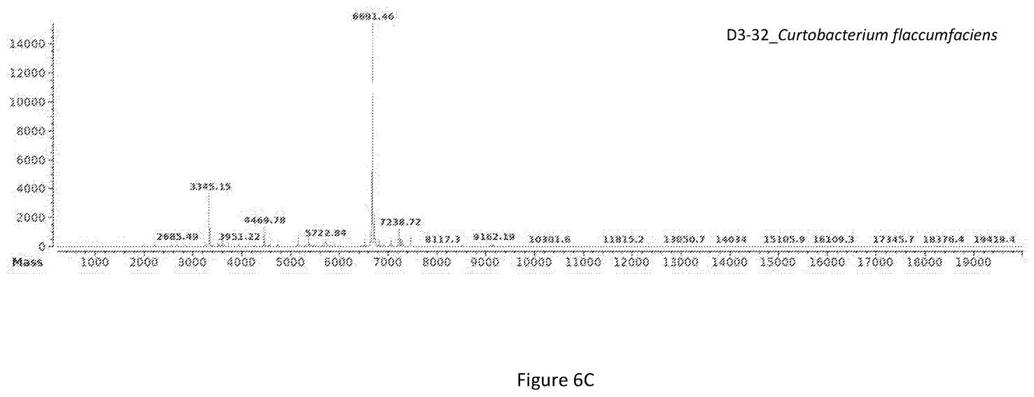

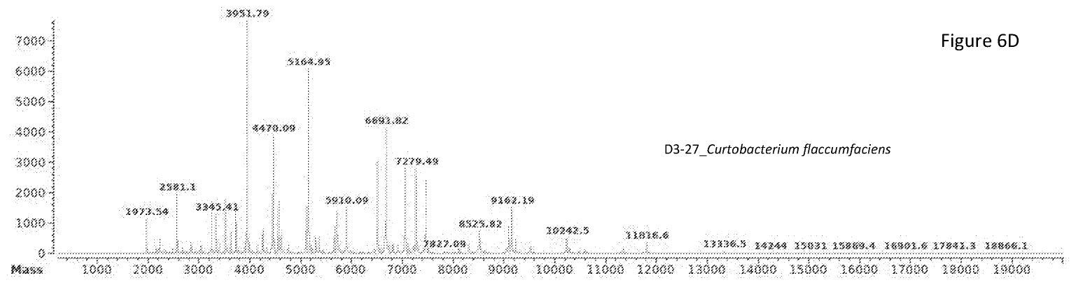

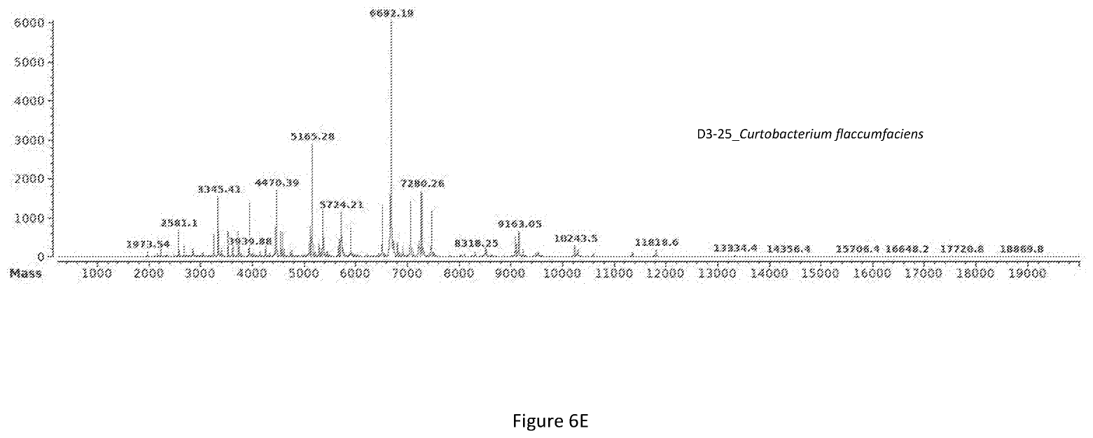

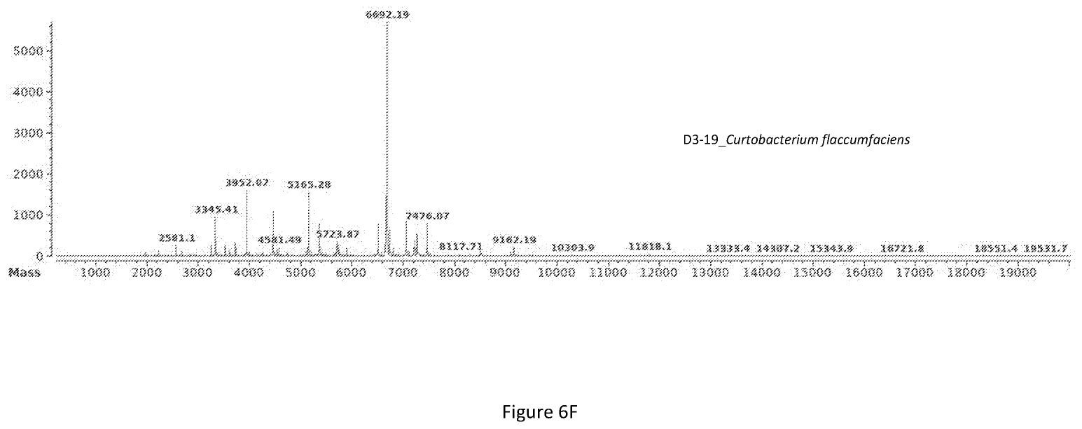

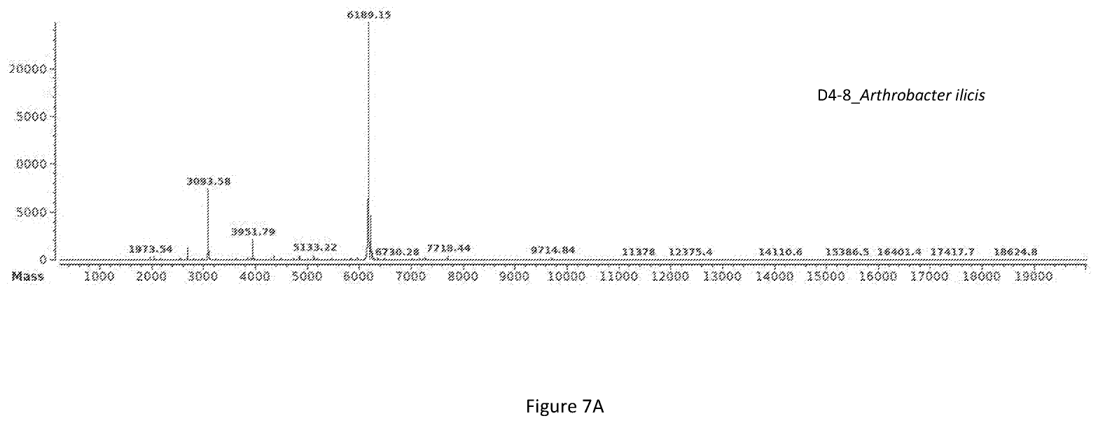

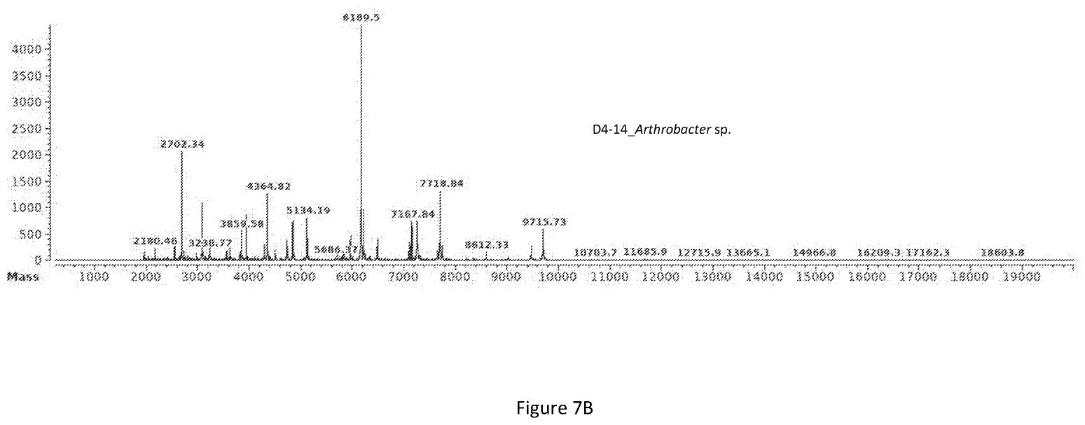

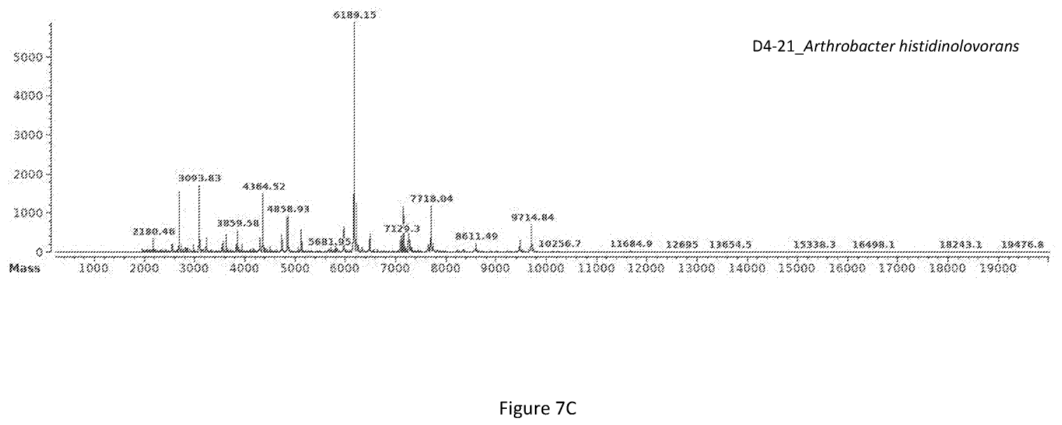

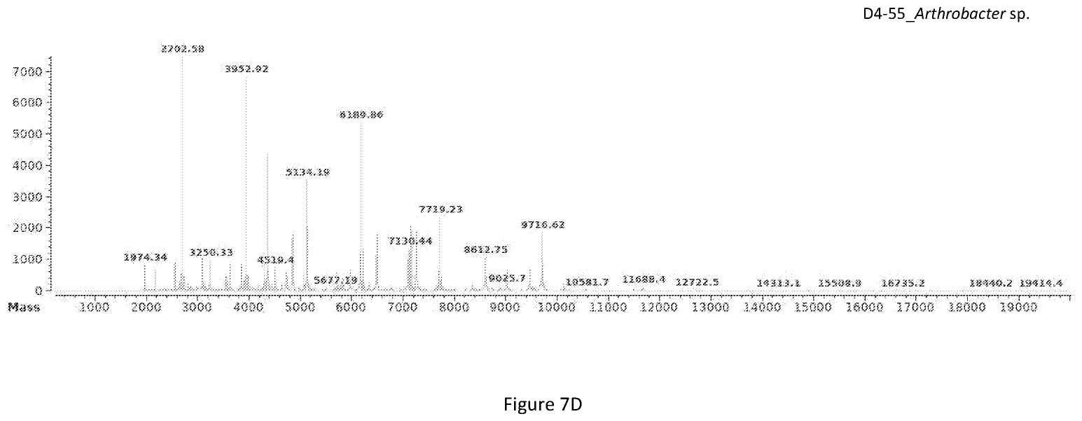

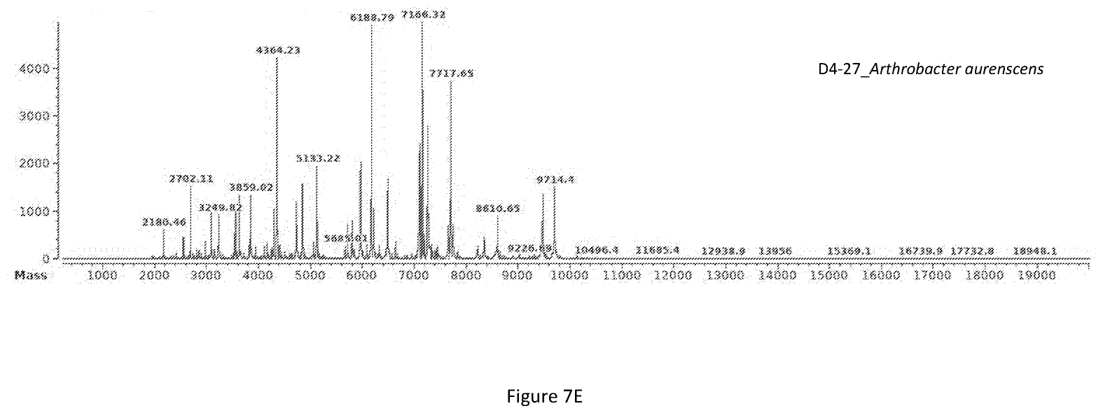

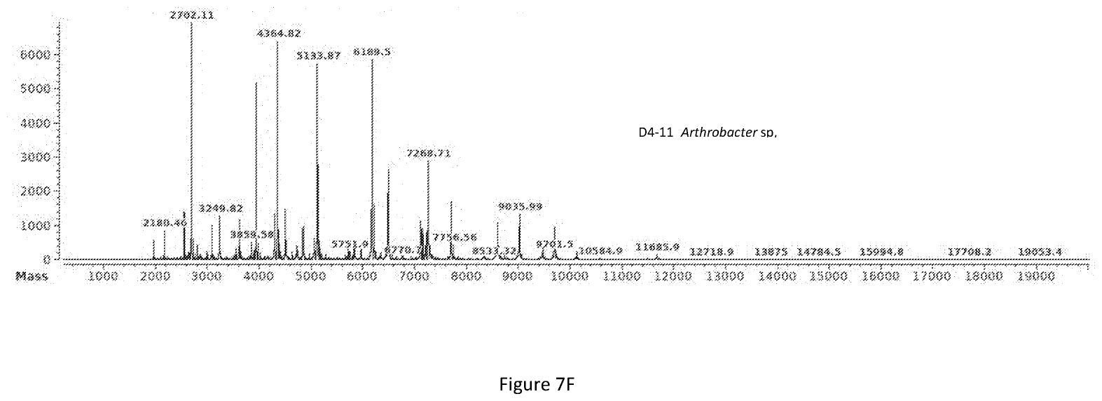

The phenogram showed the clustering of 60 Curtobacterium flaccumfaciens strains into a unique clade. These strains were isolated strictly from drought tolerant lines (line 3). An assessment of the protein spectra of Curtobacterium flaccumfaciens strains (D3-19, D3-25, D3-27, D3-32, D3-34 and D3-47) indicated that protein fingerprints were highly similar, differing only with respect to peak intensity (FIG. 6). Similarly, the phenogram showed 53 Arthrobacter sp. strains in a unique clade, with all isolated from drought tolerant lines (Line 4). An assessment of the protein spectra of Arthrobacter sp. strains (D4-8, D4-11, D4-14, D4-21, D4-27 and D4-55) indicated that the protein fingerprints were also highly similar, differing only with respect to peak intensity (FIG. 7).

### Genomics

The genome of two novel Curtobacterium flaccumfaciens strains (D3-25 and D3-27) and three novel Arthrobacter sp. strains (D4-11, D4-14, D4-55) were sequenced. These novel strains were retrieved from −80° C. glycerol storage, inoculated onto R2A plates and grown at room temperature for five days. A single colony was taken from each plate and grown in 30 mL nutrient broth (NB) and incubated at 25° C. for 24 hours at 170 cycles a minute. DNA extraction was performed using the Wizard® Genomic DNA Purification Kit (A1120, Promega). The genomes of the five novel strains were sequenced using the Oxford Nanopore Technologies (ONT) MinION platform. The DNA from was first assessed with the genomic assay on the Quantus system for integrity (average molecular weight ≥30 Kb). The sequencing library was prepared using an in-house protocol modified from the official protocols for transposases-based library preparation kits (SQK-RAD004/SQK-RBK004, ONT, Oxford, UK). All libraries were sequenced on a MinION Mk1B platform (MIN-101B) with R9.4 flow cells (FLO-MIN106) and under the control of MinKNOW software. After the sequencing run finished, the fast5 files that contain raw read signals were transferred to a separate, high performance computing Linux server for basecalling and demultiplexing using ONT's Albacore software (Version 2.3.1) with default parameters. For libraries prepared with the barcoding kit (SQK-RBK004), barcode demultiplexing was achieved during basecalling. The sequencing summary file produced by Albacore was processed by the R script minion qc (https://github.com/roblanf/minion_qc) and NanoPlot (De Coster et al. 2018) to assess the quality of each sequencing run, while Porechop (Version 0.2.3, https://github.com/rrwick/Porechop) was used to remove adapter sequences from the reads. Reads which were shorter than 300 bp were removed and the worst 5% of reads (based on quality) were discarded by using Filtlong (Version 0.2.0, https://github.com/rrwick/Filtlong).

Complete circular chromosome sequences were assembled for the two novel bacterial strains using Canu (Wood & Salzberg, 2014). The assembled genomes were then polished twice with Racon and once with ONT Medaka software (Vaser, Sovic, Nagarajan, & Sikic, 2017). The genome size for the novel Curtobacterium flaccumfaciens strains D3-25 and D3-27 were 3,788,292 bp and 3,800,049 bp, respectively (Table 1). Both strains had a GC content of 71.10%. The polished genomes of these novel bacterial strains were then annotated by Prokka to predict genes and corresponding functions (Seemann 2014). The number of genes predicted for the novel bacterial strains D3-25 and D3-27 were 3834 and 3807 genes, respectively (Table 2). The genome size for the novel Arthrobacter strains, D4-11, D4-14 and D4-55 were 4,714,936 bp, 4,721,844 bp and 4,717,699 bp respectively (Table 1). The percent GC content ranged from 62.43%-62.45%. These novel bacterial strains were annotated by Prokka (Seemann 2014) to predict genes and corresponding functions. The number of genes for the novel bacterial strains D4-11, D4-14 and D4-55 were 4,949, 4,946 and 4,901 genes respectively (Table 2).

TABLE 1
Summary of properties of the final genome sequence assembly

| Strain ID | Genome size (bp) | GC content (%) | ONT MinION Coverage |
|---|---|---|---|
| D3-25 | 3,788,292 | 71.10% | 289.7x |
| D3-27 | 3,800,049 | 71.10% | 682.3x |
| D4-11 | 4,721,844 | 62.43% | 588.8x |
| D4-14 | 4,714,936 | 62.44% | 725.1x |
| D4-55 | 4,717,699 | 62.45% | 255.8x |

TABLE 2
Summary of genome coding regions

| | Genome size | No. of | No. of | No. of | No. of | No. of |
|---|---|---|---|---|---|---|
| Strain ID | (bp) | tRNA | tmRNA | rRNA | CDS | gene |
| D3-25 | 3,788,292 | 59 | 1 | 9 | 3,765 | 3,834 |
| D3-27 | 3,800,049 | 55 | 1 | 9 | 3,742 | 3,807 |
| D4-11 | 4,721,844 | 54 | 2 | 18 | 4,872 | 4,946 |
| D4-14 | 4,714,936 | 54 | 2 | 18 | 4,875 | 4,949 |
| D4-55 | 4,717,699 | 54 | 2 | 18 | 4,827 | 4,901 |

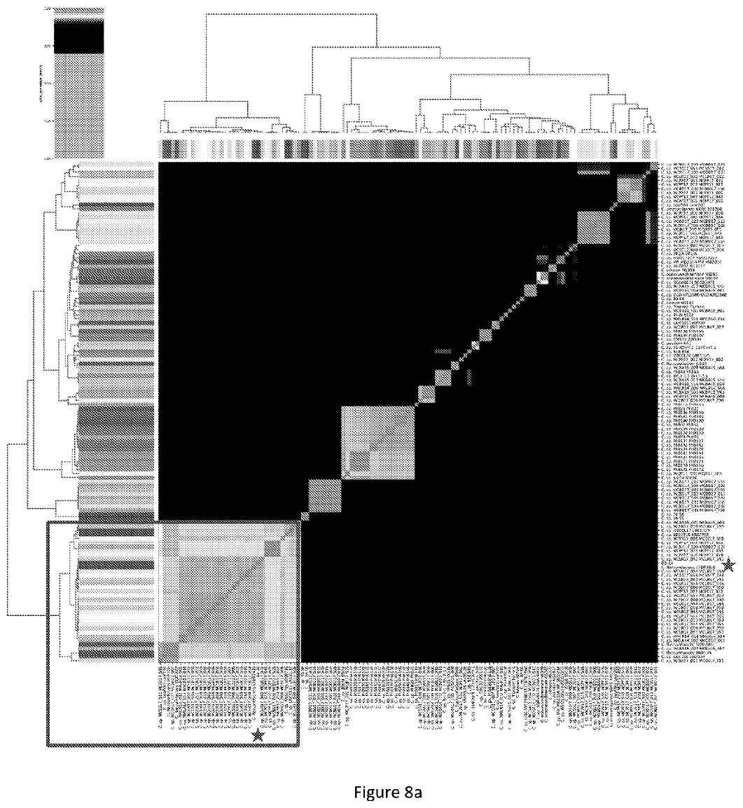

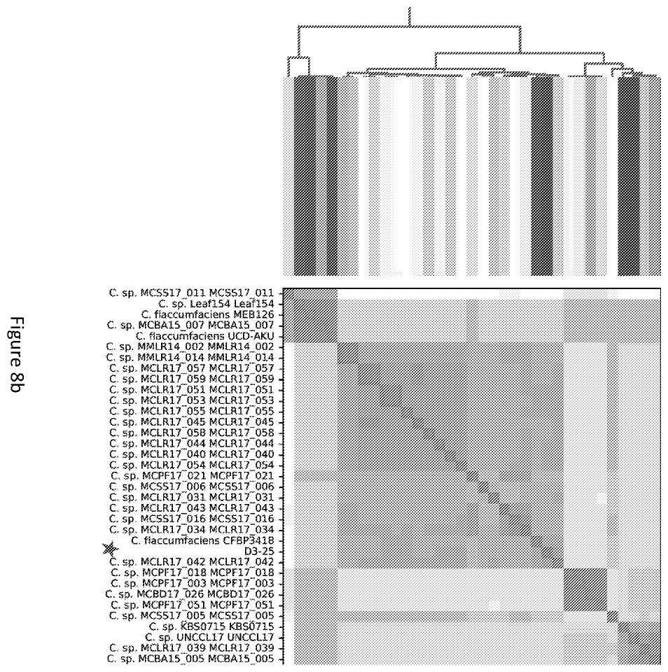

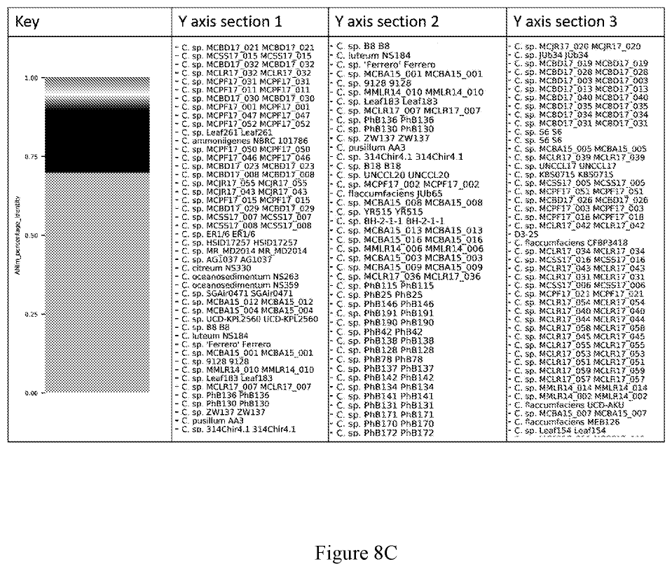

Comparative genomic analysis was performed on the C. flaccumfaciens and Arthobacter sp. against genome sequences of closely related strains available on NCBI. The average nucleotide identity (ANI) was calculated for novel C. flaccumfaciens strain D3-25 against 123 Curtobacterium sp. strains. The genome sequences were aligned and compared using minimap2 (Li 2018). Based on the ANI novel C. flaccumfaciens strain D3-25 was most similar to an environmental Curtobacterium sp. strain (MCLR17_042) and a plant pathogenic C. flaccumfaciens pv flaccumfaciens strain (CFBP3418), with an ANI of 97.68% and 97.48% respectively. On a species boundary of 95-96% (Chun et al. 2018; Richter & Rosselló-Móra 2009) C. flaccumfaciens strain D3-25 is a novel strain of the species Curtobacterium flaccumfaciens (Müller et al. 2013). A phenogram and associated heatmap was created from the ANI analysis using pyANI (Pritchard et al 2016), which represented the phylogenetic relationship between the 124 strains (FIG. 8). Novel C. flaccumfaciens strain D3-25 clustered with a group of 34 Curtobacterium sp. strains including 31 environmental Curtobacterium sp. strains and 3 Curtobacterium flaccumfaciens strains (one plant pathogen pv flaccumfaciens strain, one Arabidopsis endophyte strain, one residential environment strain).

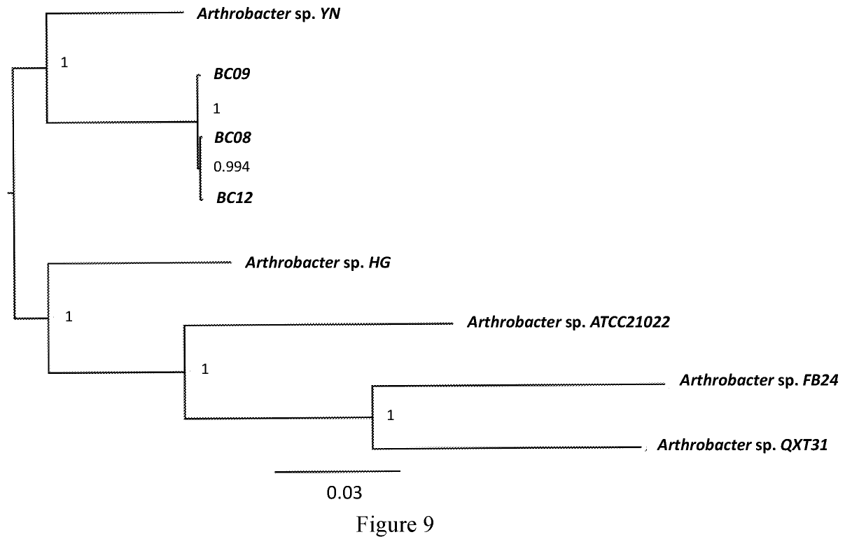

Five Arthrobacter sp. genome sequences that were publicly available on NCBI were acquired and used for pan-genome/comparative genome sequence analysis alongside novel Arthrobacter sp. strains D4-11, D4-14 and D4-55. A total of 118 genes that were shared by all 8 Arthrobacter sp. strains were identified by running Roary (Page et al. 2015). PRANK (Löytynoja 2014) was then used to perform a codon aware alignment. A maximum-likelihood phylogenetic tree (FIG. 9) was inferred using FastTree (Price, Dehal & Arkin 2010) with Jukes-Cantor Joins distances and Generalized Time-Reversible and CAT approximation model. Local support values for branches were calculated using 1000 resamples with the Shimodaira-Hasegawa test.

Novel Arthrobacter sp. strains D4-11, D4-14 and D4-55 formed a tight clade, adjacent to Arthrobacter sp. strain YN, suggesting a phylogenetic relationship between these two bacterial strains. Moreover, these clades were separated from other clades with a strong local support value (100%). This separation supports that bacterial strains D4-11, D4-14 and D4-55 are novel and from a novel Arthrobacter species. The ANI was calculated for novel Arthrobacter sp strains D4-11, D4-14 and D4-55 against 5 Arthrobacter sp. strains. Based on the ANI novel Arthrobacter sp. strains D4-11, D4-14 and D4-55 were most similar to an environmental Arthrobacter sp. strain (YN), with an ANI of 87.82%, while the three strains had an ANI of 99.93-99.95% (FIG. 9)

### EXAMPLE 5

### Genome Sequence Features Supporting the Endophytic Niche and Beneficial Traits of the Curtobacterium flaccumfaciens and Arthrobacter sp. Novel Bacterial Strains

The genome sequences of Curtobacterium flaccumfaciens novel strains D3-25 and D3-27, along with Arthrobacter sp. novel strains D4-11, D4-14 and D4-55 were assessed for the presence/absence of genes associated with phytohormone production which have been shown to be important for bacteria conferring enhanced growth and drought tolerance of host plants (Li et al, 2018; Barnawal et al 2017). In addition, the genome of D3-25 was compared to Curtobacterium flaccumfaciens p.v. flaccumfaciens strain CFBP3418 (plant pathogen) and assessed for the presence/absence of genes associated with pathogenicity. The annotated genomes were analysed using KEGG via sequence homology searches against genes associated with major metabolic pathways, and in particular pathways showing genes associated with phytohormone production, stress tolerance and pathogenicity. Presence of genes associated with phytohormone production and an absence of genes associated with pathogenicity supports Curtobacterium flaccumfaciens novel strains D3-25 and D3-27, and Arthrobacter sp. novel strains D4-11, D4-14 and D4-55 having an endophytic niche and beneficial traits.

### Growth Promotion and Drought Tolerance

The genomes of Curtobacterium flaccumfaciens novel strains D3-25 and D3-27 and Arthrobacter sp. novel strains D4-11, D4-14 and D4-55 contained genes associated with phytohormone production, and in particular indole acetic acid (IAA). An amidase enzyme (amiE) that catalyses the production of IAA from indole-3-acetamide was identified in Curtobacterium flaccumfaciens novel strains D3-25 and D3-27 and Arthrobacter sp. novel strains D4-11, D4-14 and D4-55, with all strains containing two homologues. Arthrobacter sp. novel strains D4-11, D4-14 and D4-55 also had a tryptophan 2-monooxygenase (iaaM) that catalyses the product indole-3-acetamide from tryptophan, however this was not identified in Curtobacterium flaccumfaciens novel strains D3-25 and D3-27. This suggests IAA production in Curtobacterium flaccumfaciens novel strains D3-25 and D3-27 was via indole, whereas Arthrobacter sp. novel strains D4-11, D4-14 and D4-55 was via tryptophan.

### Pathogenicity Factors

The genomes of Curtobacterium flaccumfaciens novel strains D3-25 and D3-27 had a reduced complement of genes associated with regulating pathogenicity, compared to Curtobacterium flaccumfaciens p.v. flaccumfaciens strain CFBP3418 (plant pathogen)

(Table 3). There was an absence of the SlyA transcriptional regulator that is critical for virulence in bacterial pathogens (Zou et al 2012). There was a reduced complement of genes associated with Type II and Type IV secretion systems. There was also a reduced complement of genes associated with ABC transporters, and in particular those involved in the membrane transport of lipopolysaccharides and phosphonates. These ABC transporters have been associated with pathogenicity by facilitating glycoconjugate/polysaccharide biogenesis and nutrient acquisition, respectively (Lewis et al 2012; Davidson et al 2008; Gebhard et al). Finally, there was a reduced complement of genes associated with carbohydrate metabolism, and in particular the Fructose/Mannose and Starch/Sucrose pathways, which suggests a reduced capacity to utilise the host for carbohydrate supplementation (Fatima and Senthil-Kumar 2015.

TABLE 3
Presence/absence of genes associated with pathogenicity in
Curtobacterium flaccumfaciens novel strains D3-25 and D3-27
and the pathogenic strain
Curtobacterium flaccumfaciens p.v. flaccumfaciens CFBP3418

| | | Curtobacterium |
|---|---|---|
| | Curtobacterium | flaccumfaciens p.v. |
| | flaccumfaciens | flaccumfaciens |
| | (D3-25 and D3-27) | (CFBP3418) |
| Virulence Regulation | | |
| slyA | 0 | 1 |
| Secretion Systems | | |
| Type II | 4 | 5 |
| Type IV | 2 | 3 |
| ABC Transporters | | |
| Lipopolysaccharides | 0 | 2 |
| Phosphonates | 0 | 3 |
| Carbohydrate Metabolism | | |
| Fructose/Mannose | 26 | 28 |
| Starch/Sucrose | 23 | 24 |

### EXAMPLE 6

### In Vitro Assays Supporting the Mutualistic Niche and Beneficial Traits of the Curtobacterium flaccumfaciens and Arthrobacter sp. Novel Bacterial Strains

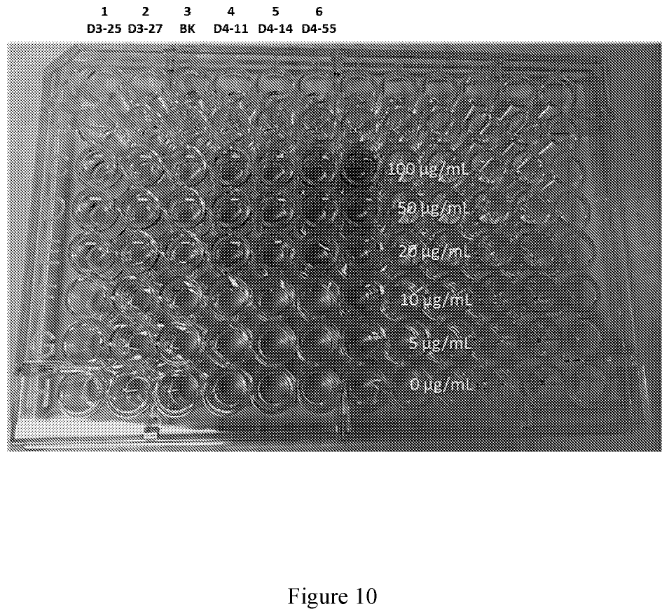

An in vitro assay was developed to assess the ability of Curtobacterium flaccumfaciens novel strains D3-25 and D3-27 and Arthrobacter sp. novel strains D4-11, D4-14 and D4-55 to produce IAA. The assay utilised the Van Urk Salkowski reagent and followed the Salkowski's method (Ehmann, 1977). The strains were grown in tryptone yeast calcium chloride (TYC) broth supplemented with 0.1% (w/v) L-tryptophan, and incubated at 28° C. for 4 days. The broth was centrifuged and the pellet was discarded. The supernatant (1 ml) was mixed with the Salkowski's reagent (2 mL: 2% 0.5 FeCl₃ in 35% HCLO₄) in a 96-well microtitre plate and incubated for 25-30 mins in the dark. Each strain had three replicates. IAA standards were also prepared at concentrations of 0, 5, 10, 20, 50 and 100 μg/mL. Arthrobacter sp. novel strains D4-11, D4-14 and D4-55 produced a visible colour change consistent with the presence of IAA (FIG. 10). The approximate concentration of IAA for the novel Arthrobacter sp. strains was 5-10 μg/mL (5000-10000 ppb). Curtobacterium flaccumfaciens novel strains D3-25 and D3-27 produced no visible colour change indicating that no IAA was produced. Given that the proposed precursor for Curtobacterium flaccumfaciens novel strains D3-25 and D3-27 is indole and not tryptophan it was expected that these strains would not produce IAA in this assay.

The concentration of IAA produced by Curtobacterium flaccumfaciens novel strains D3-25 and D3-27 and Arthrobacter sp. novel strains D4-11, D4-14 and D4-55 in the colourmetric assay (TYC broth, supplemented with 0.1% tryptophan) was quantified using a Bruker maXis HD UHR-Q-ToF (60,000 resolution) with an ESI source on-line with a UHPLC 1290 Infinity Binary LC system (Table 4). Concentrations of IAA were determined against a standard curve (0, 69, 137, 500 ppb). Arthrobacter sp. novel strains D4-11, D4-14 and D4-5 produced IAA at concentrations of 1364 ppb, 426 ppb and 1513 ppb, respectively. Curtobacterium flaccumfaciens novel strains D3-25 and D3-27 produced no detectable IAA.

TABLE 4
Quantification (ppb) of IAA in Curtobacterium flaccumfaciens novel
strains D3-25 and D3-27 and Arthrobacter sp. novel strains D4-11,
D4-14 and D4-55 from the IAA in vitro assay.

| Sample | Concentration (ppb) |
|---|---|
| Control | 0 |
| D3-25 | 0 |
| D3-27 | 0 |
| D4-11 | 1364 |
| D4-14 | 426 |
| D4-55 | 1513 |

### EXAMPLE 7

### In Planta Inoculations in Wheat and Bean Supporting Mutualistic Niche and Beneficial Traits of Arthrobacter sp. and Curtobacterium flaccumfaciens Novel Bacterial Strain

To assess direct interactions between the Curtobacterium flaccumfaciens novel strains D3-25 and D3-27 and Arthrobacter sp. novel strains D4-11, D4-14 and D4-55 and plants, an early growth assay was established in wheat and bean. A total of 6 bacterial strains (D3-25, D3-27, D4-11, D4-14, D4-55 and a related strain Bac1) were cultured in Lysogeny Broth (LB) overnight at 26° C. The following day seeds of wheat (cultivar Bob White Red Haplotype) and bean (cultivar Snap Bean) were surface-sterilised by soaking in 80% ethanol for 3 mins, then washed 5 times in sterile distilled water. The seeds were then soaked in the overnight cultures for 4 hours at 26° C. in a shaking incubator. For control seedlings, seeds were soaked in LB without bacteria for 4 hours at 26° C. in a shaking incubator. The seeds were planted into a pot trial, with three replicates (pots) per strain/control, with a randomised design. A total of 15 seeds were planted per pot to a depth of 1 cm for wheat, while 5 seeds were planted per pot to a depth of 1 cm for bean. The potting medium contained a mixture of 25% potting mix, 37.5% vermiculite and 37.5% perlite. The plants were grown for 4 weeks and then assessed for health (i.e. no disease symptoms), measured and photographed. The lengths of the longest shoot were measured. Data was statistically analysed using a one-way ANOVA and Tukey test to detect the presence of any significant difference (p≤0.05) between treatments using OriginPro 2018 (Version b9.5.1.195).

### Wheat

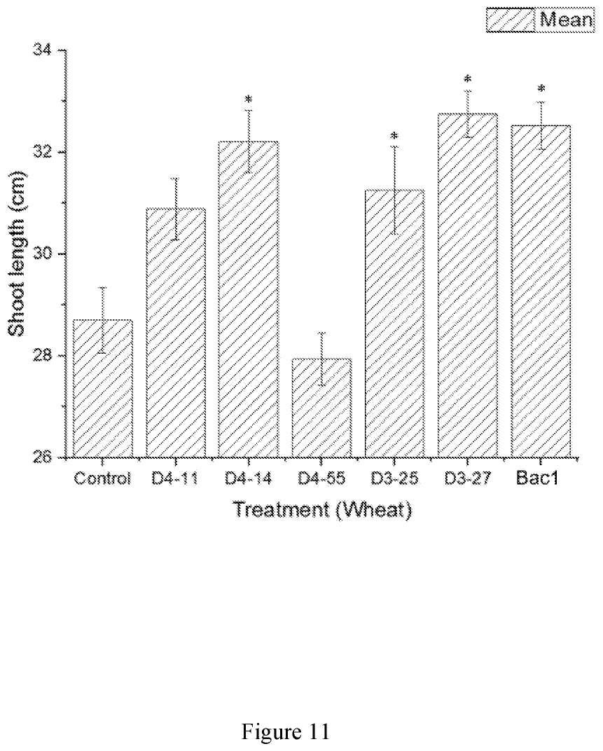

Wheat seedlings inoculated with Curtobacterium flaccumfaciens novel strains D3-25 and D3-27 and Arthrobacter sp. novel strains D4-11, D4-14 and D4-55 were healthy with no disease symptoms recorded on leaves or roots. The length of the shoots inoculated with the Curtobacterium flaccumfaciens novel strains D3-25 and D3-27 and Arthrobacter sp. novel strains D4-14 were significantly greater to the control, with a percentage increase of 8.89%, 14.09% and 12.22%, respectively (FIG. 11).

### Bean

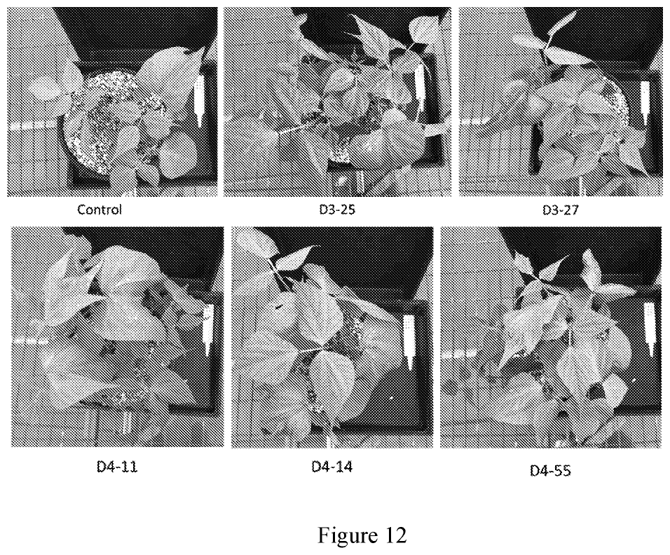

Bean seedlings inoculated with Curtobacterium flaccumfaciens novel strains D3-25 and D3-27 and Arthrobacter sp. novel strains D4-11, D4-14 and D4-55 were healthy with no disease symptoms recorded on leaves or roots (FIG. 12).

Overall, the beneficial affects of the novel bacteria on wheat, coupled with the health (no disease) both wheat and bean, suggest these bacteria are mutualistic and not pathogenic.

### EXAMPLE 8

### In Planta Inoculations in Triticeae Species Supporting Mutualistic Niche and Beneficial Traits of Arthrobacter sp. and Curtobacterium flaccumfaciens Novel Bacterial Strain

To assess the growth promotion effect of Curtobacterium flaccumfaciens novel strain D3-25 and Arthrobacter sp. novel strains D4-14 on plants, a seedling assay was established with members of the tribe Triticeae (wheat—Triticum aestivum, spelt—Triticum spelta, durum—Triticum durum, rye—Secale cereale, oats—Avena sativa, barley—Hordeum vulgare). Bacterial strains D3-25 and D4-14 were cultured in Lysogeny Broth (LB) overnight at 26° C. The following day seeds of the Triticeae species were sterilised by soaking in 80% ethanol for 3 mins, then washed 5 times in sterile distilled water. An OD reading was taken to determine the CFU/mL. The cultures were spun down and washed in PBS twice before being resuspended in their original volume of overnight culture. Cultures were then serially diluted in PBS to concentrations of 10⁻¹ (1 in 10), 10⁻² (1 in 100), 10⁻³ (1 in 1000), 10⁻⁴ (1 in 10000) and 10⁻⁵ (1 in 100000) respectively. Seeds were soaked in neat, or serial dilutions for 4 hours at 26° C. in a shaking incubator. As a control, seeds were soaked in PBS without bacteria for 4 hours at 26° C. in a shaking incubator. Fifteen inoculated seeds were then placed on moist sterile filter paper in sterile petri plates and allowed to grow for seven days.

There were four replicates per treatment. Data was statistically analysed using a one-way ANOVA and Tukey test to detect the presence of any significant difference (p≤0.05) between treatments using OriginPro 2018 (Version b9.5.1.195).

### Wheat

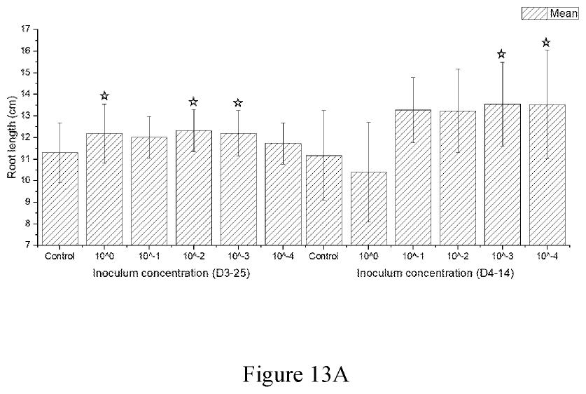

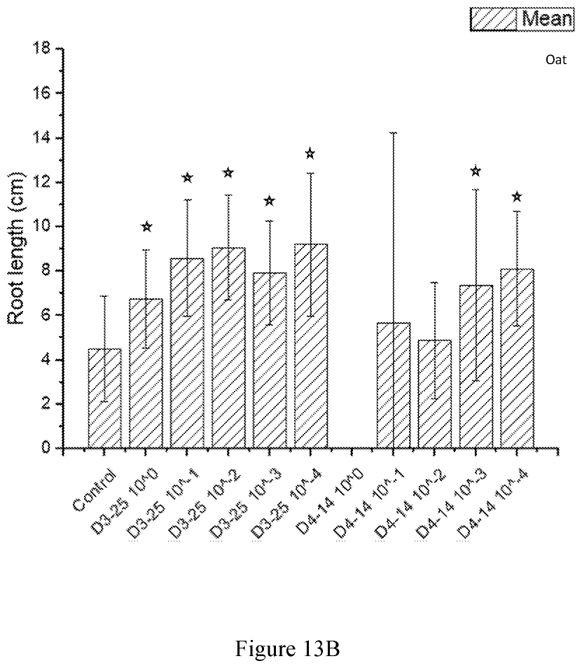

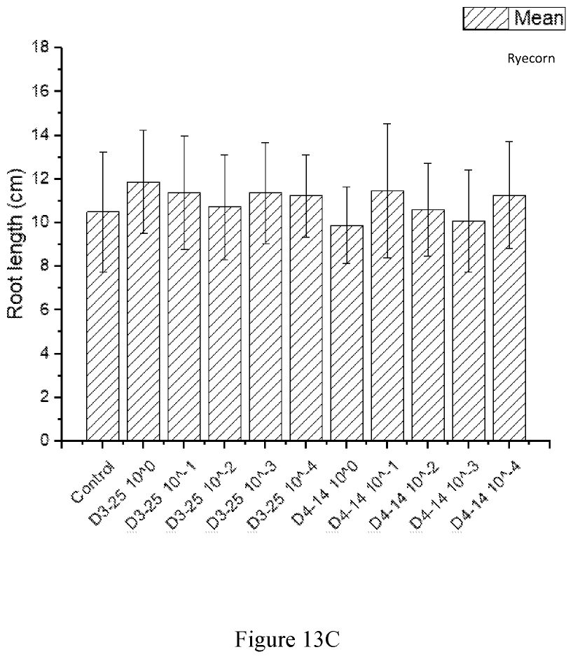

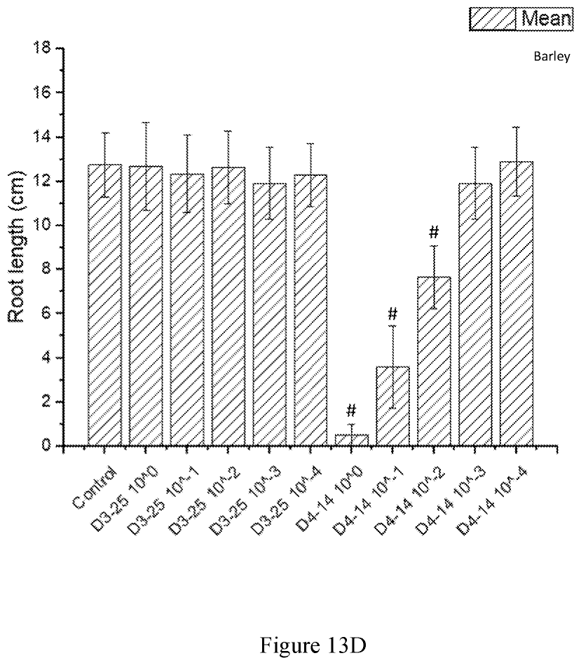

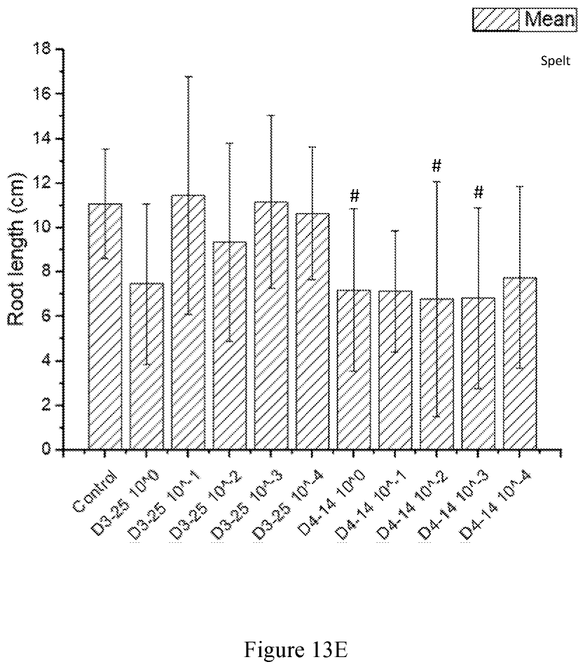

Wheat seedlings inoculated in Curtobacterium flaccumfaciens novel strain D3-25 dilutions of 10°, 10⁻² and 10⁻³ (containing 7×10⁸, 7×10⁶ and 7×10⁵ CFU/mL, respectively) had significantly longer roots than the control, with a percentage increase of 7.94%, 9.05%, 7.95% respectively. Similarly, wheat seedlings inoculated in Arthrobacter sp. novel strain D4-14 dilutions of 10⁻³ and 10⁻⁴ (containing 1.13×10⁶ and 1.13×10⁵ CFU/mL, respectively) had significantly longer roots than the control, with a percentage increase of 21.93% and 21.78% respectively. (FIG. 13A).

### Oat

Oat seedlings inoculated in Curtobacterium flaccumfaciens novel strain D3-25 solutions of 10⁻¹, 10⁻², 10⁻³ and 10⁻⁴ (containing 8.19×10⁷, 8.19×10⁶, 8.19×10⁵, 8.19×10⁴ CFU/mL, respectively) had significantly longer roots than control, with a percentage increase of 90.81%, 101.55%, 63.85% and 104.68% respectively. Similarly, oat seedlings inoculated in Arthrobacter sp. novel strain D4-14 solutions of 10⁻³ and 10⁻⁴ (5.81×10⁵ and 5.81×10⁴ CFU/mL, respectively) had significantly longer roots than control, with a percentage increase of 63.85% and 80.14% respectively. (FIG. 13B).

### Ryecorn

Ryecorn seedlings inoculated with all Curtobacterium flaccumfaciens novel strain D3-25 solutions had significantly equivalent root growth with the control. Similarly, ryecorn seedlings inoculated with all Arthrobacter sp. novel strain D4-14 solutions had significantly equivalent root growth with the control. (FIG. 13C).

### Barley

Barley seedlings inoculated with all Curtobacterium flaccumfaciens novel strain D3-25 solutions had significantly equivalent root growth with the control. In contrast, barley seedlings inoculated with 10°, 10⁻¹ and 10⁻², Arthrobacter sp. novel strain D4-14 solutions had significantly shorter root growth than the control, with a percentage decrease of 96.03%, 71.85% and 39.98%, respectively. (FIG. 13D).

### Spelt

Spelt seedlings inoculated with all Curtobacterium flaccumfaciens novel strain D3-25 solutions had significantly equivalent root growth with the control. Similarly, spelt seedlings inoculated with all Arthrobacter sp. novel strain D4-14 solutions had significantly shorter root growth than the control, with a percentage decrease of 34.99%, 38.71% and 38.28%, respectively. (FIG. 13E).

Overall, the growth promoting effect of the novel strains on wheat and oat, but not ryecorn, barley and spelt, suggest the mutualistic niche may be limited to specific species with the Triticeae family.

### EXAMPLE 9

### In Planta Inoculations in Wheat Supporting Mutualistic Niche and Drought Tolerance Activity of Arthrobacter sp. and Curtobacterium flaccumfaciens Novel Bacterial Strain

To assess the ability of Curtobacterium flaccumfaciens novel strain D3-25 and Arthrobacter sp. novel strain D4-14 to aid drought tolerance, an in planta assay was established in wheat exposed to varying levels of drought. The wheat seeds (cultivar Bob White Red Haplotype) were sterilised as per Example 8. The seeds were then soaked in overnight cultures of the two novel bacteria for 4 hours at 26° C. in a shaking incubator. For control seedlings, seeds were soaked in LB without bacteria for 4 hours at 26° C. in a shaking incubator. Seeds were planted into 20 cm diameter pots containing potting medium (25% potting mix, 37.5% vermiculite and 37.5% perlite). For each treatment, eight seeds were planted at a depth of 1 cm around the edge of each pot, with a total of 12 replicate pots per treatment. All treatments were subjected to one of three watering conditions: well-watered (300 mL water every two days), mild drought (150 mL of water every two days), or severe drought (50 mL every two days). After the first week of growth, seeds that had not germinated were removed, reducing the total number of plants per pot to four. After six weeks of growth, the plants were separated into aerial and root tissue, then weighed (wet weight). Data were analysed using OriginPro 2018 (Version b9.5.1.195) as per Example 8.

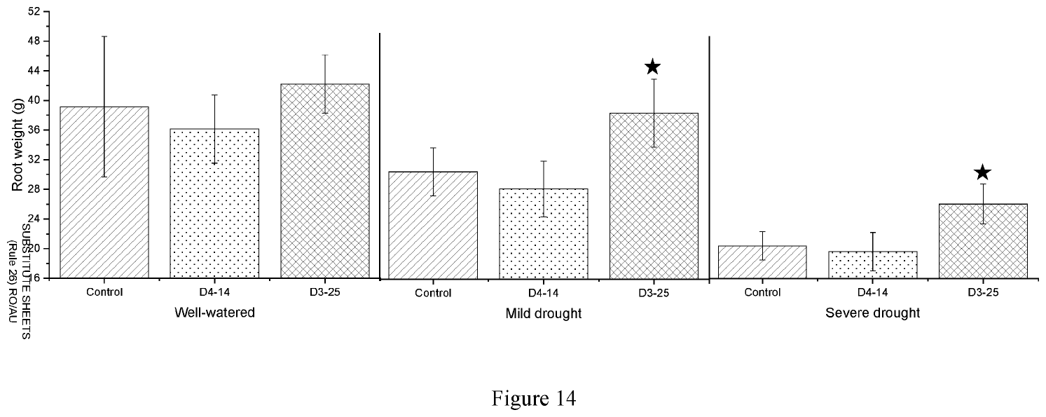

Root weight was significantly greater in 6-week old wheat plants inoculated with Curtobacterium flaccumfaciens novel strain D3-25 under mild drought and severe drought conditions, compared to the Arthrobacter sp. novel strain D4-14 and the control (FIG. 14). Curtobacterium flaccumfaciens novel strain D3-25 increased the root weight of wheat by 26.00 and 27.61% under mild drought and severe conditions, respectively. Root weight was 15.07% greater in wheat plants inoculated with Curtobacterium flaccumfaciens novel strain D3-25 under well-watered conditions, although not significant. Root weight was significantly equivalent in wheat plants inoculated with Arthrobacter sp. novel strain D4-14 and the control, under all conditions.

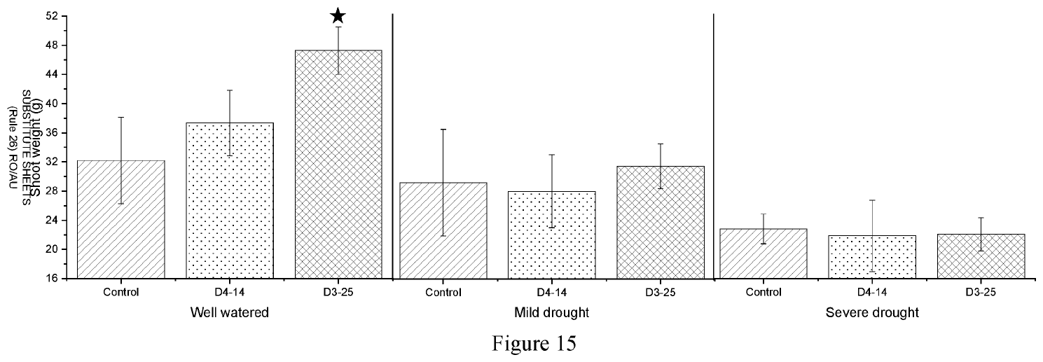

Shoot weight was significantly greater in 6-week old wheat plants inoculated with Curtobacterium flaccumfaciens novel strain D3-25 under well-watered conditions, compared to the Arthrobacter sp. novel strain D4-14 and the control (FIG. 15). Curtobacterium flaccumfaciens novel strain D3-25 increased the shoot weight of wheat by 46.82% under well-watered conditions. Shoot weight was 7.71% greater in wheat plants inoculated with Curtobacterium flaccumfaciens novel strain D3-25 under mild drought conditions, although not significant. Shoot weight was significantly equivalent in wheat plants inoculated with Arthrobacter sp. novel strain D4-14 and the control, under all conditions, although shoot weight was 15.92% greater under well-watered conditions.

Overall, the positive root growth effects observed for Curtobacterium flaccumfaciens novel strain D3-25 under drought conditions suggest this bacteria may play a key role in aiding drought tolerance in wheat.

Without being bound by any particular theory or mode of action, it is thought that the seed carries the genetics and recruited microbiome for intergenerational transmission to aid in the growth and development of the subsequent generation. If different plant lines are exposed to a stress (e.g. drought), both the genetics of the plant and the microbiome play an important role in conferring tolerance or susceptibility. Tolerant and susceptible lines can be identified through phenotyping. Profiling and isolating the seed microbiome of tolerant and susceptible lines to determine microbes that seem enriched in the tolerant lines can then aid the subsequent generation to tolerate the stress as well, particularly given that plants are sessile (i.e. they can't escape the stress relocating to a new more benign, less-stressful environment and their offspring will have enhanced survival/establishment adaptive features if colonised with stress adaptive microbes). These microbes can then be ‘re-deployed’ into a broader array of germplasm and varieties to confer the adaptive stress tolerance ‘trait’.

Finally, it is to be understood that various alterations, modifications and/or additions may be made without departing from the spirit of the present invention as outlined herein.

### REFERENCES

1. Barnawal, D., Bharti, N., Pandey, S. S., Pandey, A., Chanotiya, C. S. and KaIra, A. (2017), Plant growth-promoting rhizobacteria enhance wheat salt and drought stress tolerance by altering endogenous phytohormone levels and TaCTR1/TaDREB2 expression. Physiol Plantarum, 161: 502-514. doi:10.1111/pp1.12614.

2. Chun, J, Oren, A, Ventosa, A, Christensen, H, Arahal, D R, da Costa, M S, Rooney, A P, Yi, H, Xu, X W, De Meyer, S & Trujillo, M E 2018, ‘Proposed minimal standards for the use of genome data for the taxonomy of prokaryotes’, Int J Syst Evol Microbiol, vol. 68, no. 1, pp. 461-6.

3. Davidson, A. L., Dassa, E., Orelle, C., & Chen, J. (2008). Structure, function, and evolution of bacterial ATP-binding cassette systems. Microbiology and molecular biology reviews: MMBR, 72 (2), 317-364. doi:10.1128/MMBR.00031-07.

4. De Coster, W, D'Hert, S, Schultz, D T, Cruts, M & Van Broeckhoven, C 2018, ‘NanoPack: visualizing and processing long read sequencing data’, Bioinformatics.

5. Gebhard, S., Tran, S. L., & Cook, G. M. (2006). The Phn system of Mycobacterium smegmatis: a second high-affinity ABC-transporter for phosphate. Microbiology, 152 Pt 11, 3453-65.

6. Fatima, U., & Senthil-Kumar, M. (2015). Plant and pathogen nutrient acquisition strategies. Frontiers in plant science, 6, 750. doi:10.3389/fpls.2015.00750.

7. Ehmann, A 1977 Journal of Chromatography 132 (2): 267-276.

8. Lewis, V. G., Ween, M. P. & McDevitt, C. A. Protoplasma (2012) 249: 919. https://doi.org/10.1007/s00709-011-0360-8.

9. Li, M., Guo, R., Yu, F., Chen, X., Zhao, H., Li, H., & Wu, J. (2018). Indole-3-Acetic Acid Biosynthesis Pathways in the Plant-Beneficial Bacterium Arthrobacter pascens ZZ21. International journal of molecular sciences, 19 (2), 443. doi: 10. 3390/ijm s19020443.

10. Li, H 2018, ‘Minimap2: pairwise alignment for nucleotide sequences’, Bioinformatics, pp. bty191-bty.

11. Löytynoja, A 2014, ‘Phylogeny-aware alignment with PRANK’, in D J Russell (ed.), Multiple Sequence Alignment Methods, Humana Press, Totowa, N.J., pp. 155-70, DOI 10.1007/978-1-62703-646-7_10, <https://doi.orq/10.1007/978-1-62703-646-7_10>.

12. Massela A P, Bartram A K, Truszkowski J M, Brown D G, Neufeld J D. 2012. ‘PANDAseq: PAired-eND Assembler for Illumina sequences’. BMC Bioinformatics 13 (31).

13. Müller, H, Zachow, C, Alavi, M, Tilcher, R, Krempl, P M, Thallinger, G G & Berg, G 2013, ‘Complete Genome Sequence of the Sugar Beet Endophyte Pseudomonas poae RE*1-1-14, a Disease-Suppressive Bacterium’, Genome Announc, vol. 1, no. 2, p. e0002013.

14. Page, A J, Cummins, C A, Hunt, M, Wong, V K, Reuter, S, Holden, M T, Fookes, M, Falush, D, Keane, J A & Parkhill, J 2015, ‘Roary: rapid large-scale prokaryote pan genome analysis’, Bioinformatics, vol. 31, no. 22, pp. 3691-3.

15. Price, M N, Dehal, P S & Arkin, A P 2010, ‘FastTree 2—approximately maximum-likelihood trees for large alignments’, PLoS One, vol. 5, no. 3, p. e9490.

16. Pritchard, L., Glover, R. C., Humphris, S., Elphinstone, J. G., & Toth, I. K. (2016). Genomics and taxonomy in diagnostics for food security: soft-rotting enterobacterial plant pathogens.

17. Richter, M & Rosselló-Móra, R 2009, ‘Shifting the genomic gold standard for the prokaryotic species definition’, Proceedings of the National Academy of Sciences, vol. 106, no. 45, pp. 19126-31.

18. Seemann, T 2014, ‘Prokka: rapid prokaryotic genome annotation’, Bioinformatics, vol. 30, no. 14, pp. 2068-9.

19. Vaser, R, Sovic, I, Nagarajan, N & Sikic, M 2017, ‘Fast and accurate de novo genome assembly from long uncorrected reads’, Genome Res, vol. 27, no. 5, pp. 737-46.

20. Wagner M R, Lundberg D S, Tijana G, Tringe S G, Dangl J L, Mitchell-Olds T. ‘Host genotype and age shape the leaf and root microbiomes of a wild perennial plant’. Nature Communications. 2016 Jul. 12; 7:12151.

21. Wood, D E and Salzberg S L, Genome Biology 2014 15:R46.

22. Zou, L., Zeng, Q., Lin, H., Gyaneshwar, P., Chen, G., & Yang, C. H. (2012). SlyA regulates type III secretion system (T3SS) genes in parallel with the T3SS master regulator HrpL in Dickeya dadantii 3937. Applied and environmental microbiology, 78 (8), 2888-2895. doi:10.1128/AEM.07021-11.

1-24. (canceled)

25. A substantially purified or isolated endophyte strain from a plant, wherein said endophyte is a strain of Curtobacterium flaccumfaciens and/or Arthrobacter sp., and wherein said endophyte strain provides improved environmental stress tolerance to plants into which it is inoculated.

26. The endophyte according to claim 25, wherein the environmental stress tolerance is drought stress tolerance.

27. The endophyte according to claim 25, wherein the endophyte is purified or isolated from a plant of the Poaceae family, particularly from the genus Triticum, including T. aestivium.

28. The endophyte according to claim 25, wherein the endophyte is purified or isolated from a plant that is tolerant to abiotic conditions, more particularly, drought tolerant.

29. The endophyte according to claim 25, wherein the endophyte produces one or more phytohormones, preferably the phytohormone is an auxin, more preferably the auxin is indole acetic acid (IAA).

30. The endophyte according to claim 25, wherein the endophyte is non-pathogenic.

31. The endophyte according to claim 30, wherein the non-pathogenic endophyte is absent genes associated with pathogenicity in a plant.

32. The endophyte according to claim 25, wherein the endophyte is selected from the group consisting of Curtobacterium flaccumfaciens strain D3-27 as deposited with the National Measurement Institute of 9 Jul. 2019 with accession number V19/013682, Curtobacterium flaccumfaciens strain D3-25 as deposited with the National Measurement Institute of 9 Jul. 2019 with accession number V19/013683, Arthrobacter sp. strain D4-11 as deposited with the National Measurement Institute of 9 Jul. 2019 with accession number V19/013680, Arthrobacter sp. strain D4-14 as deposited with the National Measurement Institute of 9 Jul. 2019 with accession number V19/013681, Arthrobacter sp. strain D4-55 as deposited with the National Measurement Institute of 9 Jul. 2019 with accession number V19/013684.

33. A plant or part thereof infected with one or more endophytes according to claim 25.

34. The plant or part thereof according to claim 33, wherein the plant or plant part includes an endophyte-free host plant or part thereof stably infected with said endophyte.

35. The plant or part thereof according to claim 33, wherein the plant or plant part is an agricultural plant species selected from one or more of forage grass, turf grass, bioenergy grass, grain crop and industrial crop, preferably wherein the grain crop or industrial crop grass selected from the group consisting of those belonging to the genus Triticum, including T. aestivum (wheat), those belonging to the genus Avena including A. sativa (oats) those belonging to the genus Hordeum, including H. vulgare (barley), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), those belonging to the genera Miscanthus, Paspalum, Pennisetum, Poa, Eragrostis and Agrostis.

36. A phytohormone produced by the endophyte according to claim 25.

37. The phytohormone according to claim 36, wherein the phytohormone is an auxin, preferably IAA or a derivative, isomer and/or salt thereof.

38. A method for producing a phytohormone, said method including infecting a plant with the endophyte according to claim 25, cultivating the plant under conditions suitable to produce the phytohormone and isolating the phytohormone from the plant or culture medium.

39. A method of stimulating growth of a plant or plant part, said method including:
inoculating said plant or plant part with the endophyte according to claim 25; and cultivating inoculated plant or plant parts.

40. The method of claim 39, wherein plant or plant part is cultivated under abiotic stress conditions.

41. A method for enriching a plant or plant part for endophytes conferring improved environmental stress tolerance, said method including:
cultivating plant or plant parts under environmental stress conditions;
measuring plant or plant parts to identify germplasm that is tolerant or susceptible to environmental stress conditions;
profiling and isolating endophytes from a plant or plant part that is tolerant or susceptible to environmental stress conditions identifying endophytes enriched in a plant or plant part that is tolerant to environmental stress conditions;
infecting said plant or plant part with germplasms of identified endophytes to confer improved environmental stress tolerance.

42. The method according to claim 41, wherein the cultivated plant or plant part and/or the infected plant or plant part is a seed.

43. The method according to claim 41, wherein the environmental stress tolerance is drought tolerance.

44. The method according to claim 43, wherein the endophyte(s) is the endophyte according to claim 25.
